(19) United States
(12) Patent Application Publication
(10) Pub. No.: US 2003/0008820 A1
(43) Pub. Date: Jan. 9, 2003

(54) Methods and products related to FGF dimerization

(75) Inventors: Chi-Pong Kwan, Framingham, MA (US); Ganesh Venkataraman, Waltham, MA (US); Zachary Shriver, Cambridge, MA (US); Rahul Raman, Cambridge, MA (US); Ram Sasisekharan, Cambridge, MA (US)

Correspondence Address: Helen C. Lockhart, c/o Wolf, Greenfield & Sacks, P.C., Federal Reserve Plaza, 600 Atlantic Avenue, Boston, MA, 02210-2211, US

(73) Assignee: Massachusetts Institute of Technology, Cambridge, MA (US)

(21) Appl. No.: 10/108,195

(22) Filed: Mar. 27, 2002

Related U.S. Application Data
(60) Provisional application No. 60/279,165, filed on Mar. 27, 2001.

(51) Int. Cl. A61K 38/18; C07K 14/50; C12P 21/02; C12N 5/06

(52) U.S. Cl. 514/12; 435/69.1; 435/320.1; 435/325; 530/399

(57) ABSTRACT

The invention is methods and products related to FGF dimerization. In particular compositions of FGF dimers are provided. Methods of using those compositions including therapeutic uses are also provided

### RELATED APPLICATIONS

This application claims priority under Title 35 USC §119(e) to copending U.S. Provisional Patent Application Ser. No. 60/279,165, filed Mar. 27, 2001, entitled METHODS AND PRODUCTS RELATED TO FGF DIMERIZATION which is incorporated herein by reference in its entirety.

### FIELD OF THE INVENTION

The invention relates to FGF dimers and methods of use. Pharmaceutical compositions, therapeutic utilities and screening assays are also provided.

### BACKGROUND OF THE INVENTION

Fibroblast growth factors (FGFs) are involved in a wide range of physiological processes including morphogenesis as well as disease processes such as tumor angiogenesis (Ornitz, D. M. (2000) Bioessays 22(2), 108-12; Taipale, J. et al. (1997) Faseb J 11(1), 51-9; Hanahan, D. et al. (1996) Cell 86(3), 353-64). The FGF family consists of at least 20 members including the well-characterized acidic FGF (FGF1) and basic FGF (FGF2), both of which are potent mitogens of many cell types. FGF signaling is mediated primarily through high-affinity interaction with cell-surface FGF receptors (FGFRs), transmembrane polypeptides composed of immunoglobulin-like and tyrosine kinase domains. FGF binding to different isoforms of FGFR is believed to trigger receptor dimerization followed by transphosphorylation of specific tyrosine residues (Schlessinger, J. et al. (1995) Cell 83(3), 357-60). Phosphorylated tyrosine residues in turn activate other signaling proteins, leading to cell proliferation, migration and survival.

For proper presentation to its cogent FGFR, FGF2, and other members of the FGF family, interact with heparin/heparan sulfate-like glycosaminoglycans (HLGAGs). Consisting of a disaccharide repeat of glucosamine and uronic acid, HLGAGs are heterogeneous in length (10 to 100 disaccharide units) and chemical composition (including differential sulfation, acetylation and epimerization of each disaccharide unit) (Guimond, S. et al. (1993) J Biol Chem 268(32), 23906-14). Found in the extracellular matrix and on cell surface as part of proteoglycans, HLGAGs modulate FGF2 activity by low-affinity interactions with specific FGF2 and FGFR binding sites (Faham, S. et al. (1996) Science 271(5252), 1116-20; Omitz et al. (1995) Science 268(5209), 432-6; Kan al. (1993) Science 259(5103), 1918021) facilitating FGF2 binding to FGFR. HLGAGs promote FGF2-induced activation of FGFR through a number of mechanisms, including regulating diffusion rate of FGF2 (Dowd, C. J. et al. (1999) J Biol Chem 274(8), 5236-44; Flaumenhaft, R. et al. (1990) J Cell Biol 111(4), 1651-9) and possibly dictating the specificity of FGF2-FGFR binding through interactions with both FGF2 and FGFR (Guimond, S. E. et al. (1999) Curr Biol 9(22), 1343-6; Kan, M. et al. (1999) J Biol Chem 274(22), 15947-52).

Confusion exists in the prior art concerning the status of FGF when it interacts with the FGF receptor and initiates signal transduction. Examination of apo-FGF and FGF-HLGAG crystal structures has led to the proposal of preferential FGF2 self-association in a cis mode, with substantial protein-protein interactions between the adjacent molecules (Venkataraman, G. et al. (1996) Proc Natl Acad Sci USA 93(2), 845-50). However, NMR studies predict a different mode of FGF oligomerization, viz., a symmetrical FGF2 dimer with possible disulfide bond formation between two surface cysteines (Moy, F. J. et al. (1997) Biochemistry 36(16), 4782-91. Furthermore, the recently solved FGF1-decasaccharide co-crystal points to a FGF trans dimer involving no FGF-FGF contacts (DiGabriele, A. D. et al. (1998) Nature 393(6687), 812-7, a mechanism for dimerization which may or may not extend to other members of the FGF family, viz., FGF2. More recently, several crystallographic studies of FGF-FGFR and FGF-FGFR-HLGAG complexes, including FGF2: FGFR1 (Plotnikov, A. N. et al. (1999) Cell 98(5), 641-50) FGF1: FGFR2 (Plotnikov, A. N. et al. (2000) Cell 101(4), 413-24), FGF2: FGFR2 (Plotnikov, A. N. et al. (2000) Cell 101(4), 413-24), FGF1: FGFR2 (Stauber, D. J. et al. (2000) Proc Natl Acad Sci USA 97(1), 49-54), reveal assemblages of two FGFs bound to two FGFRs with no FGF-FGF contacts in the complex. Thus, conflicting biochemical and biophysical evidence makes it unclear whether FGF oligomerization is important for signaling through FGFR and, if so, which dimerization mode of FGF, involving either protein contact or no protein contact, mediates FGF signaling. This problem is compounded when one considers that the two recent crystal structures of the ternary complex between FGF, FGFR, and HLGAG (Schlessinger, J. et al. (2000) Mol Cell 6(3), 743-50; Pellegrini, L. et al. (2000) Nature 407(6807), 1029-34) reveal different stoichiometries for the complex with markedly divergent geometries.

### SUMMARY OF THE INVENTION

It was discovered according to some aspects of the invention that FGF dimers are biologically active and result in transphosphorylation of FGFR. Prior art studies have demonstrated that HLGAGs facilitate FGF oligomerization (Omitz, D. M. et al. (1992) Mol Cell Biol 12(1), 240-7; Herr, A. B. et al. (1997) J Biol Chem 272(26), 16382-9; Spivak-Kroizman, T. et al. (1994) Cell 79(6), 1015-24) in vitro. Due to a lack of direct evidence, however, it was unclear whether this biochemical phenomenon was important for FGF2 signaling. Furthermore, different modes of FGF-FGF interactions have been observed in various studies, drawing into question what modes of FGF oligomerization, if any, are biologically relevant. Using conformational studies and molecular engineering techniques to systematically explore proposed modes of FGF2 oligomerization and to evaluate the importance of FGF-FGF interactions in signaling, it was discovered according to the invention that dimerization of FGF is important for the biological activity of FGF. The data described herein demonstrates that a FGF dimer involving substantial non-covalent protein-protein contact is readily formed and it is able to mediate signaling.

In some aspects the invention provides a pharmaceutical composition of a modified FGF dimer comprising two FGF monomers linked to one another, wherein the dimer includes at least one modification from a native FGF dimer, and a pharmaceutically acceptable carrier. In other aspects the invention is a composition of a stabilized modified FGF dimer comprising two FGF monomers linked to one another, wherein the dimer includes at least one modification from a native FGF dimer.

In some embodiments the FGF dimer of the pharmaceutical composition is stabilized. In other embodiments the pharmaceutical composition is sterile.

In some embodiments the two FGF monomers are FGF2. In preferred embodiments the modification is a linker molecule connecting the two monomers and more preferably the linker molecule is a peptide. The FGF dimer in some embodiments is a protein produced by recombinant DNA technology, e.g., by expression of a nucleic acid having the sequence of SEQ ID NO.: 5 or a functional equivalent. In other embodiments at least one FGF monomer has an amino acid sequence corresponding to SEQ ID NO.: 1 or a functional variant thereof. Optionally the peptide linker is GAL, GAR, or GARG. In some embodiments the peptide linker includes a protease site or an integrin binding sequence, such as RGD.

In other embodiments the modification is in at least one of the FGF monomers and is a cysteine residue that does not occur in the native FGF monomer. For instance, at least one FGF monomer may have an amino acid sequence corresponding to SEQ ID NO.: 7 or a functionally equivalent variant thereof, but wherein the FGF monomer includes at least one cysteine residue at amino acid number 81 (SEQ ID NO.: 2). Alternatively, the pharmaceutical composition includes at least one FGF monomer has an amino acid sequence corresponding to SEQ ID NO.: 7 or a functionally equivalent variant thereof, but wherein the FGF monomer includes at least one cysteine residue at amino acid number 100 (SEQ ID NO.: 3). In some embodiments the pharmaceutical composition includes both FGF monomers having an amino acid sequence corresponding to SEQ ID NO.: 7 or a functionally equivalent variant thereof, but wherein the FGF monomers include at least one cysteine residue at each of amino acid numbers 81 and 100 (SEQ ID NO.: 4). Optionally at least one of the naturally occurring cysteines includes a conservative or non-conservative substitution. In yet other embodiments both of the FGF monomers include a cysteine residue that does not occur in the native FGF monomer.

Thus, the composition may include an FGF dimer having at least one FGF monomer with an amino acid sequence corresponding to SEQ ID NO.: 2, SEQ ID NO.: 3, or SEQ ID NO.: 4.

The two FGF monomers are linked to one another by a chemical linkage such as for example a disulfide bond.

In other embodiments the modification of the FGF dimer is in at least one of the FGF monomers and is a deletion of at least one or all of the 9 N-terminal amino acid residues of the monomer. This deletion may be in one or both of the monomers. The N-terminal end of the monomer may also be substituted with a protease site or an integrin binding sequence.

Optionally the dimer may be complexed with an HLGAG and or the FGF dimer may be formulated in a microparticle.

In another aspect the invention is a FGF dimer composed of two FGF monomers linked to one another via a peptide linker, optionally formulated in a pharmaceutically acceptable carrier. In some embodiments the dimer is complexed with an HLGAG. In other embodiments at least one FGF monomer has an amino acid sequence corresponding to SEQ ID NO.: 1 or a functionally equivalent variant thereof.

The peptide linker may be of a variety of lengths or sequences. Some preferred linkers include but are not limited to GAL, GAR, and GARG. Optionally the peptide linker includes a protease site or an integrin binding sequence, such as RGD.

The invention in other aspects is a method for promoting signal transduction, by contacting a cell with an FGF dimer of any one of claims 1-25 or 28-34 in an effective amount for promoting signal transduction.

In other aspects the invention relates to therapeutic methods, such as a method for treating stroke, promoting angiogenesis, promoting collateral blood vessel formation, promoting nerve regeneration, promoting wound healing, treating or preventing a nervous system disease, i.e. a central nervous system disease or a peripheral nervous system disease, or preventing myocardial damage in heart disease and surgery. The methods are performed by administering to a subject in need thereof, a stabilized FGF dimer composed of two FGF monomers linked to one another or other FGF dimer of the invention, and a pharmaceutically acceptable carrier in an effective amount for treating the disorder or obtaining the desired biological effect. Preferably the FGF dimer is in the form of any of the pharmaceutical compositions described herein. In some embodiments the subject is a human. In other embodiments the FGF dimer is pre-incubated with an HLGAG prior to administering it to the subject.

In other aspects, the invention is a method for treating or preventing an FGF sensistive disorder by administering to a subject in need thereof, an effective amount for activating an FGFR a stabilized FGF dimer composed of two FGF monomers linked to one another or other FGF dimer of the invention.

In yet other aspects the invention is a screening assay for identifying an FGF dimer binding compound, by contacting a library of compounds with the FGF dimer of any one of the invention, and identifying a compound that binds the FGF dimer to identify the FGF dimer binding compound. Optionally the method includes the step of determining whether the FGF binding compound is an FGF inhibitor by determining whether the FGF binding compound can block FGF dimer interaction with an FGF receptor.

In other aspects the invention relates to compositions of the FGF dimer binding compound or the FGF inhibitor identified according to the assay and methods for inhibiting FGF activity in a subject by administering to the subject an FGF inhibitor.

In other aspects the invention relates to therapeutic methods using an FGF inhibitor, such as a method for treating cancer, inhibiting angiogenesis, or treating chronic inflammation. These methods are also performed by administering to a subject in need thereof, the FGF inhibitor of the invention, and a pharmaceutically acceptable carrier in an effective amount for treating the disorder or obtaining the desired biological effect. In some embodiments the subject is a human.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 depicts the analysis of various binding sites on FGF2. The surface of a FGF2 molecule can be approximated as the faces of a parallelepiped. Of the six faces, two opposite faces represent the receptor binding sites (pointing into and out of the plane of the paper), while the other four (denoted as oligomerizing and heparin binding) represent directions about which FGF can associate. Two of the three oligomerizing directions are aligned along the same plane. Translation of FGF2 molecules along these two directions forms the basis of FGF2 oligomerization.

FIG. 2 illustrates the proposed modes of FGF dimerization. Either a closed or an open triangle is drawn inside each FGF molecule to distinguish different orientations. The round indentation within FGF represents the heparin-binding domain. HLGAG is depicted as a chain of beads. (A) Two FGF molecules, oriented asymmetrically in cis, bind to the same side of HLGAG in a “side-by-side” fashion (Herr, A. B. et al. (1997) J Biol Chem 272(26), 16382-9; Venkataraman, G. et al. (1996) Proc Natl, Acad Sci USA 93(2), 845-50; Venkataraman, G. et al. (1999) Proc Natl Acad Sci USA 96(5), 1892-7). (B) Two FGF molecules are oriented in trans to the axis of HLGAG in a “head-to-head” fashion (DiGabriele, A. D. et al. (1998) Nature 393(6687), 812-7). (C) Four FGF molecules interact both in cis and trans with HLGAG (Moy, F. J. et al. (1997) Biochemistry 36(16), 4782-91). Note that, for the cis interaction, the two FGF molecules are symmetrically related as opposed to the dimer in (A).

FIG. 3 details the oxidative crosslinking studies. (A) Oxidative crosslinking of wild-type FGF2 and cysteine mutant. Wild-type FGF2 was oxidized with (lane 1) or without (lane 2) heparin. A minor amount of dimer was detected, which likely resulted from the crosslinking reaction between unfolded protein. Cysteine mutant, which was designed based on the model of FGF2 dimerization (Venkataraman, G. et al. (1996) Proc Natl Acad Sci USA 93(2), 845-50), was oxidized with (lane 3) or without (lane 4) heparin under the same conditions as the wild-type. All reaction products were separated using non-reducing SDS-PAGE (15%) followed by silver staining. The extent of oligomerization achieved by the cysteine mutant was compared to wild-type. (B) Schematic representation of the protein-protein and protein-HLGAG interactions in cysteine mutants. Two cysteine mutant molecules are shown, each with two dimer interfaces as represented by striped (site p) and open (site p′) rectangles. Two solvent-exposed cysteines (C81 and C100 as shown near site p′ and p, respectively) were engineered such that they would position in close proximity with each other at the interface. (C) Dimerization and oligomerization of cysteine mutant were mediated by the native structure of the protein. Lane 1, cysteine mutant alone; lane 2, cysteine mutant oxidized without heparin; lane 3, same as lane 2 but protein was heat/SDS-denatured prior to oxidative crosslinking and lane 4, same as lane 2 but treated with 1 mM DTT. Oxidative crosslinking of cysteine mutant was abolished by either denaturing or reducing treatments.

FIG. 4 illustrates the engineering, cloning and purification of dFGF2. (A) A scheme is shown for linking two FGF2 genes and subcloning them into an expression vector for protein expression. Restriction sites (NdeI, SacI and SpeI) were introduced to the 5′ and 3′ ends of FGF2 cDNA by PCR. (B) Restriction digest of the expression vector with two tandemly-linked FGF2 cDNAs is shown. Lane 1, NdeI/SpeI digest of the expression vector; lane 2, NdeI/SacI digest and lane 3, SacI/SpeI digest. (C) Schematic of the protein product obtained upon expression of the genetic construct of (A). An N-terminus His tag, a C-terminus T7 tag and two thrombin cleavage sequences (gray rectangles) are present to facilitate protein purification. The arrows indicate the positions of thrombin cleavage. (D) Wild-type mFGF2 (lane 1) and dFGF2 (lane 2) are separated by SDS-PAGE under reducing condition. The molecular size is shown on the side.

FIG. 5 shows the structural properties of dFGF2. The near UV CD spectrum of dFGF2 is shown. dFGF2 was concentrated to 1 μM and buffer-exchanged into 10 mM sodium phosphate, pH 7.2. Data were recorded in an average of 20 scans between 195 nm and 260 nm. The characteristic intense negative CD signals observed near 200 nm is indicative of properly folded FGF2.

FIG. 6 describes the competitive binding of dFGF2 for FGFR2. (A) MALDI-MS profile of a mixture of wild-type FGF2 and the ectodomain of FGFR2. Observed in the mass spectrum are (M+H)⁺ ion for an FGF2 dimer (m/z 30,214) and trimer (m/z 45,132), FGFR2 monomer (m/z 24,888) and dimer (m/z 49,572), and a 1:1 FGF2-FGFR2 complex (m/z 39,896). The theoretical molecular masses for FGF2 and FGFR2 are 15114 and 24864, respectively. (B) Mass spectrum of the FGF2/FGFR2 mixture in the presence of a homogenous HLGAG decasaccharide. Addition of a decasaccharide (Deca) to FGF2/FGFR2 promotes the formation of a 2:2 FGF2:FGFR2 complex with an observed (M+H)⁺ ion at m/z 82,650 (with Deca) or m/z 79,872 (without Deca). The (M+H)⁺ ion for two dimeric FGFR2 species are also observed, the first at m/z 49,692 represents the apo complex and the second at m/z 52,474 is a 2:1 FGFR2:Deca complex. Inset, mass spectrum of dFGF2 added to the mixture of Deca/FGF2/FGFR2 shown above. Three high molecular weight complexes are observed: 2:2 FGF2:FGFR2 complexes with or without Deca and a 1:2 dFGF2:FGFR2 complex without Deca.

FIG. 7 illustrates the SMC proliferation assay. Serum-starved SMC were stimulated with the indicated molar concentrations of wild-type (●) and dFGF2 (∇). SMC were grown (A) in the absence of chlorate or (B) upon addition of 75 MM chlorate. After 21 h at 37° C., [³H] thymidine was added for 3 h. Cells were harvested, washed and measured [³H] thymidine incorporation was counted. Maximal count/min for wild-type and dFGF2 were about 6000 and 5000, respectively. The proliferation curve of dFGF2 is shifted towards the left of wild-type. The molar concentrations for half-maximal proliferation by wild-type and dFGF2 are 270 pM and 60 pM, respectively.

FIG. 8 describes the HUVEC survival assay. Serum-starved HUVEC were stimulated with the indicated concentrations of wild-type and dFGF2, or without any growth factor. Cells supplemented with 10% FCS served as positive control. After 18 h, cell viability was determined colorimetrically using MTS reagent. Both wild-type and dFGF2 restored HUVEC viability following serum starvation and dFGF2 achieved the same levels of cell viability at a lower molar concentration than wild-type.

FIG. 9 details the in vivo potency of dFGF2. Slit lamp photographs of rat corneas on day 6 after implantation with Hydron pellets containing (A) no bFGF as control, (B) 1.5 pmole mFGF2, (C) 6.0 pmole mFGF2, or (D) 0.7 pmole dFGF2. Area of pellet implantation is designated with an arrow. The control pellet did not induce a significant angiogenic response, while pellets containing dFGF2 induced an intense neovascular response originating from the limbal vessels and reaching the pellet on day 6 after the implantation. Pellets containing mFGF2 (B, C) induced a less vigorous, but still detectable, angiogenic response on day 6 after implantation. In the Table, the extent of corneal angiogenic response was expressed as linear length and circumferential clock hours. * indicates Standard Error.

### DETAILED DESCRIPTION

The invention relates to biologically active FGF dimers and uses thereof. It has been discovered according the invention that FGF dimers are biologically active. The FGF dimers have, in some aspects, greatly enhanced biological activities. Most of the prior art studies describing the therapeutic use of FGF have described the use of FGF monomers. In addition, prior art studies have suggested that monomer forms of FGF2 may form active signaling complexes (Pantoliano, M. W. et al. (1994) Biochemistry 33(34), 10229-48; Pye, D. A. et al. (1999) J Biol Chem 274(19), 13456-61). For instance in a recent study, it was found that covalently linked complexes of monomer FGF with a pool of heparin dodecasaccharides were able to promote cell proliferation in vitro (Pye, D. A. et al. (1999) J Biol Chem 274(19), 13456-61). However, as observed herein (data presented in Examples section), this complex was less active than uncomplexed FGF in promoting ³H-thymidine incorporation. In contrast, the dimeric FGF (dFGF) construct presented in this study is several times more potent in biological assays than is wild-type FGF, with reduced dependence on exogenous HLGAGs for activity. The invention is based at least in part on the finding that dimers of FGF have significantly improved biological activities as compared to the monomer.

Several signaling pathways mediated by growth factors and cytokines involve binding of ligands to their cell surface receptors to facilitate receptor dimerization (Heldin, C. H. (1995) Cell 80(2), 213-23), a key step leading towards activation of intracellular signaling cascade. The structure, conformation, and oligomerization status of FGF as it interacts with FGFR to produce a biological signal are unknown. The studies of the invention have identified important characteristics of the FGF-FGFR interaction that have led to the development of a therapeutically important class of compounds. In general, it was discovered that FGF2 does have a preference to oligomerize, and the studies described herein point to the fact that this oligomerization interface involves protein-protein contact. Additionally, dimeric FGF (dFGF) constructs based on these biochemical findings were found to have potent biological activity. Thus, FGF dimers are potent mediators of FGFR dimerization and concomitant signaling.

Through rational design of a disulfide-mediated sequential dimer (cysteine mutant) based on extensive analysis of FGF2 crystal structures we demonstrated (A) a marked increase in the amount of oligomers formed compared with wild-type FGF2, which has the same number of surface cysteines but at different positions, (B) higher extent of oligomerization by pre-incubating cysteine mutant with heparin, and (C) that the observed oligomers involve specific protein contacts and are disulfide-mediated. The above findings strongly support a model in which FGF2 molecules self-associate through specific FGF-FGF interactions in a sequential fashion and that HLGAG may serve to provide a “platform” to stabilize the intermolecular interactions between FGF2 molecules.

To determine whether the active FGF2 dimer involves protein-protein contact in contrast to the FGF2 dimer observed in the FGF-FGFR co-crystal structures that lack protein-protein contact, a tandemly-linked dimeric FGF2 (dFGF2) molecule was constructed using conformational studies and genetic engineering tools. dFGF2 was designed such that the short distance between the two FGF2 molecules within the dimeric protein would allow for substantial FGF-FGF interactions while making the non-contacting dimer mode less favorable and therefore enable us to dissect whether a contacting FGF2 dimer can elicit biological activity. We showed though mass spectrometry that dFGF2 interacts with FGFR in a ratio of 1:2 suggesting that dFGF2 can bind to a dimer of FGFR. Furthermore, these results indicate that one mode, involving substantial protein contact, by which FGF2 and its receptor may interact is through the binding of FGFR to a FGF2 dimer. These biochemical findings were supported by the biological activity of the dFGF2 molecule, described in the Examples.

To test whether a contacting FGF2 dimer can elicit biological activity, dFGF2 was subjected to two independent cell culture assays. From both the SMC proliferation and HUVEC survival assays, dFGF2 exhibited elevated biological activity compared with wild-type FGF2. This effect was especially pronounced in the SMC assays where dFGF2 was several fold more active than wild-type and only 30% less active in the absence of HLGAGs as in their presence (as opposed to wild type FGF2 wherein activity was significantly reduced in the absence of cell surface HLGAGs). These findings demonstrated that dFGF2, in which FGF-FGF interactions are predicted to be substantial, forms an active signaling complex with the receptor. In addition, proliferation of chlorate-treated SMC demonstrated that dFGF2 was less HLGAG-dependent for signaling. These data suggest that one mechanism by which HLGAGs modulate FGF2 activity is by stabilizing two FGF2 molecules in a dimer mode to facilitate receptor dimerization. Because dFGF2 is already dimeric, its dependency on HLGAGs for proper presentation to the receptor was lower compared to wild-type FGF2. The dFGF construct was also found to be a potent pro-angiogenic agent in vivo, much more so than wild-type FGF, thus providing compelling evidence that the dFGF construct, involving substantial protein-protein contact, forms an active signaling complex at the cell surface.

Thus the biochemical, cell culture, and in vivo assays demonstrate that a FGF2 dimer is involved in the active signaling complex and are inconsistent with prior art data on the different FGF2-FGFR crystal structures, which show no FGF-FGF interactions. Such an inconsistency may reflect the inherent complexity and the multifaceted nature of the FGF system. One possible explanation is that the different structural configurations of FGF-FGFR may reflect the different states, viz., “on” or “off” states of the signaling complex. Thus, a mode of FGF2 dimerization involving protein-protein interactions could lead to a cooperative FGF2-FGFR interaction by promoting subsequent oligomerization and signaling whereas the non-contacting FGF2 dimerization may lead to an inactive complex.

Thus in some aspects, the invention relates to compositions of FGF dimers. An “FGF dimer” as used herein is an FGF dimer composed of two FGF monomers linked to one another. An FGF dimer is also referred to herein as dFGF. FGF dimers include modified FGF dimers and native FGF dimers that have been stabilized to maintain the dimeric state.

Fibroblast growth factor (FGF) was first described by its activity derived from bovine brain or pituitary tissue which was mitogenic for fibroblasts and endothelial cells. It was later noted that the primary mitogen from brain was different from that isolated from pituitary. These two factors were named acidic and basic FGF (now known as FGF 1 and FGF2), respectively, because they had similar biological activities but differed in their isoelectric points.

It is now known that a large family of proteins exist, which are considered to be FGF. The fibroblast growth factor (FGF) family consists of at least twenty three distinct members which generally act as mitogens for a broad spectrum of cell types. For example, FGF2 is mitogenic in vitro for endothelial cells, vascular smooth muscle cells, fibroblasts, and generally for cells of mesoderm or neuroectoderm origin, including cardiac and skeletal myocytes (Gospodarowicz et al., J Cell. Biol. 70:395-405, 1976; Gospodarowicz et al., J Cell. Biol. 89:568-578, 1981 and Kardami, J Mol. Cell. Biochem. 92:124-134, 1990). In vivo, FGF2 has been shown to play a role in avian cardiac development (Sugi et al., Dev. Biol. 168:567-574, 1995 and Mima et al., Proc. Nat'l. Acad. Sci. 92:467-471, 1995), and to induce coronary collateral development in dogs (Lazarous et al., Circulation 94:1074-1082, 1996). In addition to eliciting a mitogenic response that stimulates cell growth, fibroblast growth factors can stimulate a large number of cell types to respond in a non-mitogenic manner. These activities include promotion of cell migration into wound areas (chemotaxis), initiation of new blood vessel formulation (angiogenesis), modulation of nerve regeneration and survival (neurotrophism), modulation of endocrine functions, stimulation or suppression of specific cellular protein expression, extracellular matrix production and cell survival (Baird, A., and Bohlen, P., Handbook of Exp. Pharmacol. 95(1): 369-418, Springer, 1990). These properties provide a basis for using fibroblast growth factors in therapeutic approaches to accelerate wound healing, nerve repair, collateral blood vessel formation, and the like. For example, fibroblast growth factors have been suggested to minimize myocardium damage in heart disease and surgery (U.S. Pat. No. 4,378,347 to Franco).

All the members of the FGF family bind heparin and retain structural homology across species, suggesting a conservation of their structure/function relationship (Ornitz et al., J Biol. Chem. 271(25):15292-15297, 1996.). A protein is a member of the FGF family, as used herein, if it shows significant sequence and three-dimensional structural homology to other members of the FGF family, FGF-like activity in in vitro or in vivo assays and binds to heparin or heparin-like substances.

FGF signaling is mediated primarily through high-affinity interaction with cell surface FGF receptors (FGFRs), transmembrane polypeptides composed of immunoglobulin-like and tyrosine kinase domains. FGF binding to different isoforms of FGFR is believed to trigger receptor dimerization followed by transphosphorylation of specific tyrosine residues. Phosphorylated tyrosine residues in turn activate other signaling proteins, leading to cell proliferation, migration and survival. We have analyzed various crystal structures of FGF extensively and have proposed a model of FGF signaling. In this model, two molecules of FGF2 are associated preferentially along the 3 1A axis and heparin saccharide can bind to the FGF2 to stabilize the dimer. Another mode of dimerization (along the 33A axis) is also proposed.

A preferred FGF according to the invention is FGF2, and in some embodiments human FGF2 is preferred. The term “FGF2” as used herein refers to any fibroblast growth factor-2 exhibiting biologic activity. FGF2 include but are not limited to the 155 amino acid protein recognized as native FGF2 (SEQ ID NO.: 1), truncated forms exhibiting activity, extended forms such as placental FGF, higher molecular weight N-terminally extended forms and functionally equivalent FGF2 derivatives of any of these. The term specifically includes natural FGF2 extracted from mammalian tissue as well as recombinant polypeptides expressed from DNA from any species.

The three-dimensional structures of FGF2 has been determined (Eriksson, E. A., et al., Proc. Nat. Acad. Sci. U.S.A. 88: 3441-3445 (1991), Zhang, J, et al., Proc. Nat. Acad. Sci. USA. 88: 3446-3450 (1991), and Zhu, H, et al., Science 251: 90-93 (1991)). The overall structure of FGF2 can be described as a trigonal pyramid where each of the three sides are built of two β-strands together forming a β-sheet barrel of six antiparallel strands (Eriksson, E. A., et al., Proc. Nat. Acad. Sci. U.S.A. 88: 3441-3445 (1991)). The base of the pyramid is built of six additional βstrands extending from the three sides of the pyramid to close one end of the barrel for a total of twelve-strands. Thus, a threefold repeat is observed in the folding of the polypeptide chain and a pseudo-three-fold axis passes through the center of the base of the molecule and extends through the apex of the pyramid. Of the amino acids conserved within the FGF family of proteins, most are located within the core β-strand regions of FGF2.

A “modified FGF dimer” as used herein is an FGF dimer composed of two FGF monomers linked to one another, wherein the dimer includes at least one modification from a native FGF dimer. The modification may be within the amino acid sequence of one or both the FGF monomers or it may be the linkage itself. For instance, the modified FGF dimer may be composed of two naturally occurring FGF monomers which are linked by a linker molecule.

In some embodiments the modified FGF dimer is stabilized. A stabilized dimer is one in which the monomers have a higher probability of remaining in a dimeric complex than monomeric FGF ordinarily would remain in a dimeric complex. The stabilized dimer may be accomplished through a variety of mechanisms. For example a linker molecule may be used to stabilize the dimeric structure of FGF. Covalent or other non-covalent interactions may also be used to stabilize the dimer, as long as the interactions form a more stable dimeric form of FGF than the non-covalent interactions between native FGF monomers.

It was surprisingly discovered according to the invention that the stabilized FGF dimers have improved activity over FGF monomers or native dimers.

As used herein, “linked” or “linkage” means two entities are bound to one another by any physiochemical means. It is important that the linkage be of such a nature that it does not impair substantially the effectiveness of the FGF monomers or the binding specificity of the dimer with the FGFR. Keeping these parameters in mind, any linkage known to those of ordinary skill in the art may be employed, covalent or noncovalent. Linkages according to the invention include linker molecules and chemical linkages. Such means and methods of linkage are well known to those of ordinary skill in the art.

Linked monomers of FGF in an FGF dimer, when used with respect to a pharmaceutical composition of an FGF dimer refers to the fact that at least greater than 50% of the FGF monomers in the composition are in a dimeric state. Preferably at least 55%, 60%, 65%, 70%, 75%, 80%, 85%, 90%, 95%, or 100% of the FGF monomers are in a dimeric form.

A “linker molecule” as used herein is a molecule which forms an indirect linkage between the two monomers. In some embodiments the linker molecule is a spacer molecule that is attached to each of the monomers, either covalently or non-covalently. One method for attaching a spacer to the monomers is with the use of functionalized groups on the monomer to facilitate linkage and/or linker groups interposed between the monomers to facilitate their linkage. Another method involves the synthesis in a single process of both monomers and the linker, whereby the components of the dimer could be regarded as one in the same entity. For example, using recombinant DNA methodology a nucleic acid construct encoding both monomers and a linking peptide, oriented such that when the protein is expressed the linking peptide connects the two monomers, can be used to generate the dimer. These and other methods for indirect linkage are intended to be embraced by the present invention.

Specific examples of covalent bonds include those wherein bifinctional cross-linker molecules are used. The cross-linker molecules may be homobifunctional or heterobifunctional, depending upon the nature of the molecules to be conjugated. Homobifinctional cross-linkers have two identical reactive groups. Heterobifunctional cross-linkers have two different reactive groups that allow sequential conjugation reaction. Various types of commercially available cross-linkers are reactive with one or more of the following groups: primary amines, secondary amines, sulfhydriles, carboxyls, carbonyls and carbohydrates. The linker molecule may also be attached to the monomer using non-covalent bonds. Non-covalent conjugation may be accomplished by direct or indirect means including hydrophobic interaction, ionic interaction, and other affinity interactions. The linking molecules may also be modified such that they are noncleavable in physiological environments or cleavable in physiological environments. Such molecules may resist degradation.

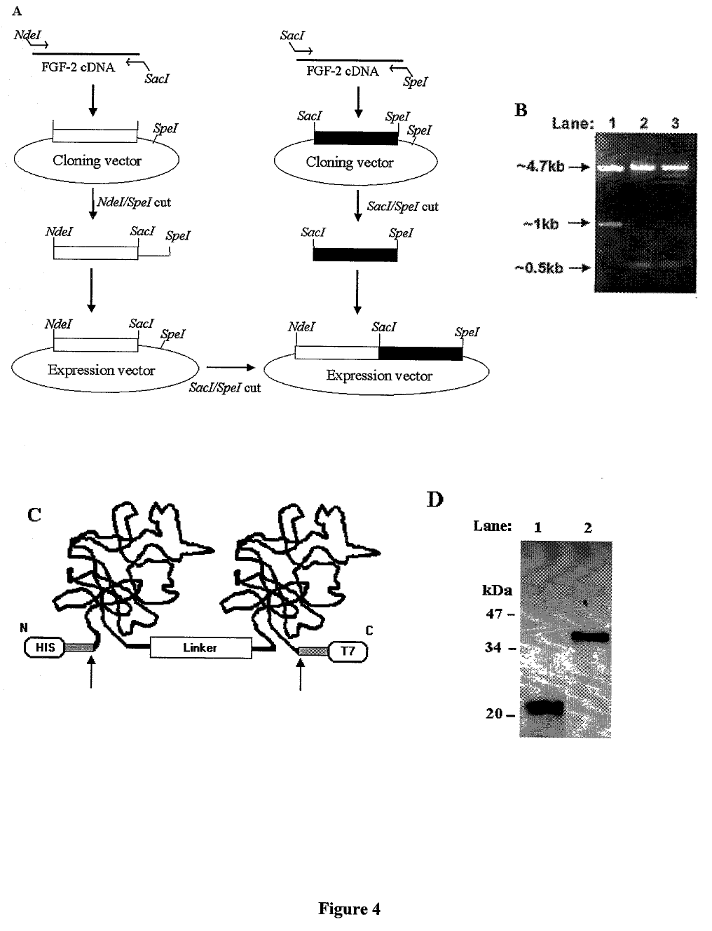

In a preferred embodiment the linker molecule is a peptide which is produced using recombinant technology along with the FGF monomers. An example of an FGF dimer produced by this method is set forth in the Examples section. The exemplary FGF dimer has the amino acid sequence of SEQ ID NO.: 6. The FGF dimer was expressed from the DNA having the sequence of SEQ ID NO.: 5. Briefly, an expression vector which will express the FGF dimer is generated. The expression vector includes the sequence for two FGF monomers and a linker peptide, operably arranged to produce a functional fusion protein. This is depicted schematically in FIG. 4. One example of a linker useful for generating the dimers is GAL. Other linkers include but are not limited to GAR and GARG. The distance of the GAL linker between the N terminus of one monomer and the C terminus of the other monomer is 27 Å. The distance between the 2 monomers of the FGF observed in crystal structures in ˜42 Å. The distance between monomers in an FGF1 dimer in transform is ˜70 Å. For FGF2 27 Åis preferred.

Thus, one of ordinary skill in the art, in light of the present disclosure, is enabled to produce the FGF dimers by standard technology, including recombinant technology, direct synthesis, mutagenesis, etc. For instance, using recombinant technology one may substitute appropriate codons in SEQ ID NO: 5 to produce the desired amino acid substitutions by standard site-directed mutagenesis techniques. Obviously, one may also use any sequence which differs from SEQ ID NO: 5 only due to the degeneracy of the genetic code as the starting point for site directed mutagenesis. The mutated nucleic acid sequence may then be ligated into an appropriate expression vector and expressed in a host such as E. coli. The resultant modified FGF dimer may then be purified by techniques well known in the art, including those disclosed below in the Examples. Preferably the FGF dimers are substantially pure. As used herein, the term “substantially pure” means that the proteins are essentially free of other substances to an extent practical and appropriate for their intended use. In particular, the proteins are sufficiently pure and are sufficiently free from other biological constituents of their hosts cells so as to be useful in, for example, protein sequencing, or producing pharmaceutical preparations.

In another set of embodiments an isolated nucleic acid encoding the modified FGF dimer of the invention is provided. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

As used herein, a coding sequence and regulatory sequences are said to be “operably joined” when they are covalently linked in such a way as to place the expression or transcription of the coding sequence under the influence or control of the regulatory sequences. In order that the coding sequences be translated into a functional protein the coding sequences are operably joined to regulatory sequences. Two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence, and the like. Especially, such 5′ non-transcribing regulatory sequences will include a promoter region which includes a promoter sequence for transcriptional control of the operably joined gene. Promoters may be constitutive or inducible. Regulatory sequences may also include enhancer sequences or upstream activator sequences, as desired.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium, or just a single time per host as the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., 62-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

As used herein, the term “stringent conditions” refers to parameters known to those skilled in the art. One example of stringent conditions is hybridization at 65° C. in hybridization buffer (3.5×SSC, 0.02% Ficoll, 0.02% polyvinyl pyrolidone, 0.02% bovine serum albumin (BSA), 25 mM NaH₂PO₄ (pH7), 0.5% SDS, 2 mM EDTA). SSC is 0.15 M sodium chloride/0.15 M sodium citrate, pH7; SDS is sodium dodecylsulphate; and EDTA is ethylene diamine tetra acetic acid. There are other conditions, reagents, and so forth which can be used, which result in the same degree of stringency. A skilled artisan will be familiar with such conditions, and thus they are not given here. The skilled artisan also is familiar with the methodology for screening cells for expression of such molecules, which then are routinely isolated, followed by isolation of the pertinent nucleic acid. Thus, homologs and alleles of the modified FGF dimer of the invention, as well as nucleic acids encoding the same, may be obtained routinely, and the invention is not intended to be limited to the specific sequences disclosed.

For prokaryotic systems, plasmid vectors that contain replication sites and control sequences derived from a species compatible with the host may be used. Examples of suitable plasmid vectors include pBR322, pUC18, pUC19 and the like; suitable phage or bacteriophage vectors include λgt10, λgt11 and the like; and suitable virus vectors include pMAM-neo, pKRC and the like. Preferably, the selected vector of the present invention has the capacity to autonomously replicate in the selected host cell. Useful prokaryotic hosts include bacteria such as E. coli, Flavobacterium heparinum, Bacillus, Streptomyces, Pseudomonas, Salmonella, Serratia, and the like.

To express the modified FGF dimer of the invention in a prokaryotic cell, it is necessary to operably join the nucleic acid sequences of the monomers and the linker to a functional prokaryotic promoter. Such promoter may be either constitutive or, more preferably, regulatable (i.e., inducible or derepressible). Examples of constitutive promoters include the int promoter of bacteriophage λ, the bla promoter of the β-lactamase gene sequence of pBR322, and the CAT promoter of the chloramphenicol acetyl transferase gene sequence of pPR325, and the like. Examples of inducible prokaryotic promoters include the major right and left promoters of bacteriophage λ (P_{L} and P_{R}), the trp, recA, lacZ, lacI, and gal promoters of E. coli, the α-amylase (Ulmanen et al., J Bacteriol. 162:176-182 (1985)) and the ζ-28-specific promoters of B. subtilis (Gilman et al., Gene sequence 32:11-20 (1984)), the promoters of the bacteriophages of Bacillus (Gryczan, In: The Molecular Biology of the Bacilli, Academic Press, Inc., NY (1982)), and Streptomyces promoters (Ward et al., Mol. Gen. Genet. 203:468-478 (1986)).

Prokaryotic promoters are reviewed by Glick (J Ind. Microbiol. 1:277-282 (1987)); Cenatiempo (Biochimie 68:505-516 (1986)); and Gottesman (Ann. Rev. Genet. 18:415-442 (1984)).

Proper expression in a prokaryotic cell also requires the presence of a ribosome binding site upstream of the encoding sequence. Such ribosome binding sites are disclosed, for example, by Gold et al. (Ann. Rev. Microbiol. 35:365-404 (1981)).

Because prokaryotic cells will not produce the modified FGF dimer of the invention with normal eukaryotic glycosylation, expression of the modified FGF dimer of the invention by eukaryotic hosts is possible when glycosylation is desired. Preferred eukaryotic hosts include, for example, yeast, fungi, insect cells, and mammalian cells, either in vivo or in tissue culture. Mammalian cells which may be useful as hosts include HeLa cells, cells of fibroblast origin such as VERO or CHO-K1, or cells of lymphoid origin, such as the hybridoma SP2/0-AG14 or the myeloma P3×63Sg8, and their derivatives. Preferred mammalian host cells include SP2/0 and J558L, as well as neuroblastoma cell lines such as IMR 332 that may provide better capacities for correct post-translational processing. Embryonic cells and mature cells of a transplantable organ also are useful according to some aspects of the invention.

In addition, plant cells are also available as hosts, and control sequences compatible with plant cells are available, such as the nopaline synthase promoter and polyadenylation signal sequences.

Another preferred host is an insect cell, for example in Drosophila larvae. When using insect cells as hosts, the Drosophila alcohol dehydrogenase promoter can be used (Rubin, Science 240:1453-1459 (1988)). Alternatively, baculovirus vectors can be engineered to express large amounts of the modified FGF dimer of the invention in insects cells (Jasny, Science 238:1653 (1987); Miller et al., In: Genetic Engineering (1986), Setlow, J. K., et al., eds., Plenum, Vol. 8, pp. 277-297).

Any of a series of yeast gene sequence expression systems which incorporate promoter and termination elements from the genes coding for glycolytic enzymes and which are produced in large quantities when the yeast are grown in media rich in glucose may also be utilized. Known glycolytic gene sequences can also provide very efficient transcriptional control signals. Yeast provide substantial advantages in that they can also carry out post-translational peptide modifications. A number of recombinant DNA strategies exist which utilize strong promoter sequences and high copy number plasmids which can be utilized for production of the desired proteins in yeast. Yeast recognize leader sequences on cloned mammalian gene sequence products and secrete peptides bearing leader sequences (i.e., pre-peptides).

A wide variety of transcriptional and translational regulatory sequences may be employed, depending upon the nature of the host. The transcriptional and translational regulatory signals may be derived from viral sources, such as adenovirus, bovine papilloma virus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or which are subject to chemical (such as metabolite) regulation.

As discussed above, expression of the modified FGF dimer of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J Mol Appl Gen. 1:273-288 (1982)); the TK promoter of Herpes virus (McKnight, Cell 31:355-365 (1982)); the SV40 early promoter (Benoist et al., Nature (London) 290:304-310 (1981)); the yeast gal4 gene sequence promoter (Johnston et al., Proc. Natl. Acad. Sci. (USA) 79:6971-6975 (1982); Silver et al., Proc. Natl. Acad. Sci. (USA) 81:5951-5955 (1984)).

As is widely known, translation of eukaryotic mRNA is initiated at the codon which encodes the first methionine. For this reason, it is preferable to ensure that the linkage between a eukaryotic promoter and the DNA sequences which encode the modified FGF dimer of the invention does not contain any intervening codons which are capable of encoding a methionine (i.e., AUG). The presence of such codons results either in the formation of a fusion protein (if the AUG codon is in the same reading frame as the modified FGF dimer coding sequence) or a frame-shift mutation (if the AUG codon is not in the same reading frame as the modified FGF dimer coding sequence).

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the expression vector. The marker may, for example, provide for prototrophy to an auxotrophic host or may confer biocide resistance to, e.g., antibiotics, heavy metals, or the like. The selectable marker gene sequence can either be directly linked to the DNA gene sequences to be expressed or introduced into the same cell by co-transfection. Additional elements may also be needed for optimal synthesis of the FGF mRNA. These elements may include splice signals, as well as transcription promoters, enhancers, and termination signals. cDNA expression vectors incorporating such elements include those described by Okayama, Molec. Cell. Biol. 3:280 (1983).

In a preferred embodiment, the introduced sequence will be incorporated into a plasmid or viral vector capable of autonomous replication in the recipient host. Any of a wide variety of vectors may be employed for this purpose. Factors of importance in selecting a particular plasmid or viral vector include the following: the ease with which recipient cells that contain the vector may be recognized and selected from those recipient cells which do not contain the vector, the number of copies of the vector which are desired in a particular host and whether it is desirable to be able to “shuttle” the vector between host cells of different species. Preferred prokaryotic vectors include plasmids such as those capable of replication in E. coli (such as, for example, pBR322, Co1E1, pSC 101, pACYC 184, and πVX. Such plasmids are, for example, disclosed by Sambrook, et al. (Molecular Cloning: A Laboratory Manual, second edition, edited by Sambrook, Fritsch, & Maniatis, Cold Spring Harbor Laboratory, 1989)). Bacillus plasmids include pC194, pC221, pT127 and the like. Such plasmids are disclosed by Gryczan (In: The Molecular Biology of the Bacilli, Academic Press, NY (1982), pp. 307-329). Suitable Streptomyces plasmids include pIJ101 (Kendall et al., J Bacteriol. 169:4177-4183 (1987)), and streptomyces bacteriophages such as φC31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704 (1986)), and Izaki (Jpn. J Bacteriol. 33:729-742 (1978)).

Preferred eukaryotic plasmids include, for example, BPV, EBV, SV40, 2-micron circle, and the like, or their derivatives. Such plasmids are well known in the art (Botstein et al., Miami Wntr. Symp. 19:265-274 (1982); Broach, In: The Molecular Biology of the Yeast Saccharomyces: Life Cycle and Inheritance, Cold Spring Harbor Laboratory, Cold Spring Harbor, N.Y., p. 445-470 (1981); Broach, Cell 28:203-204 (1982); Bollon et al., J Clin. Hematol. Oncol. 10:39-48 (1980); Maniatis, In: Cell Biology: A Comprehensive Treatise, Vol. 3, Gene Sequence Expression, Academic Press, NY, pp. 563-608 (1980)). Other preferred eukaryotic vectors are viral vectors. For example, and not by way of limitation, the pox virus, herpes virus, adenovirus and various retroviruses may be employed. The viral vectors may include either DNA or RNA viruses to cause expression of the insert DNA or insert RNA. Additionally, DNA or RNA encoding the modified FGF dimer polypeptides may be directly injected into cells or may be impelled through cell membranes after being adhered to microparticles.

Once the vector or DNA sequence containing the construct(s) has been prepared for expression, the DNA construct(s) may be introduced into an appropriate host cell by any of a variety of suitable means, i.e., transformation, transfection, conjugation, protoplast fusion, electroporation, calcium phosphate-precipitation, direct microinjection, and the like. After the introduction of the vector, recipient cells are grown in a selective medium, which selects for the growth of vector-containing cells. Expression of the cloned gene sequence(s) results in the production of the modified FGF dimer. This can take place in the transformed cells as such, or following the induction of these cells to differentiate (for example, by administration of bromodeoxyuracil to neuroblastoma cells or the like).

In some embodiments the modified FGF dimers are composed of truncated FGF monomers. For instance one or more amino acids may be removed from the N-terminal end of the protein without altering the protein folding or activity of the protein. A detailed analysis of specific sites and regions within the FGF monomers that can be manipulated is presented in Table 1. Based on the information presented in Table 1 it is possible to construct mutants of the monomers that are used for generating the dimeric FGF. The mutants can have altered biological activity, stabilization, etc.

TABLE 1
Manipulable Sites and Regions within FGF

| Name of FGF mutants | Functions |
|---|---|
| del 9 | 1^{st} 9 N-terminal aa truncation |
| del 28 | 1^{st} 28 N-terminal aa truncation |
| N102R | Promote dimerization (31 A axis) |
| L98E | ″ |
| L98E/N102R | ″ |
| R60I | ″ |
| L98E/N102R/R60I | ″ |
| Y124R | Inhibit dimerization (31 A axis) |
| L52E | Promote dimerization (33 A axis) |
| P49E | ″ |
| V68R | Inhibit dimerization (33 A axis) |
| N71R | ″ |
| Q134C | disulfide dimer (33 A axis) |
| Q134C/C87S | exclusive disulfide dimer |
| | (33 A axis) |
| R81C/S100C | disulfide dimer (31 A axis) |
| R81C/S100C/C87S/C69S | exclusive disulfide dimer |
| | (31 A axis) |
| R81C/S100C/C87S/C69S/C25S/C92S | disulfide dimer w/o internal cys |
| C87S/C69S/C25S/C92S | no cys |
| C87S/C69S/C25S/C92S/R81C | disulfide dimer w/1 cys (81C) |
| C87S/C69S/C25S/C92S/S100C | disulfide dimer w/1 cys (100C) |
| N102R/R60I | Promote dimerization (31 A axis) |
| N102R/K86A | |
| K26A | Reduce heparin binding |
| K26S | ″ |
| K125A | ″ |
| K125D | ″ |
| K119E | ″ |
| R120T | ″ |
| K119A/R120A | ″ |
| Y103A | Reduce receptor binding |
| Y111A/W114A | ″ |

For example it is possible to promote dimerization through non-covalent interactions using N102R, L98E etc. mutants. These mutants are designed to form non-covalent dimers stabilized by ionic interaction between adjacent proteins. The mutated residues are positioned at the ‘dimerization interface’ for stabilizing the dimer. Additionally dimerization may be promoted using covalent disulfide linkages e.g., R81C/S100C/C87S/C69S or cys mutant which is designed to form covalent dimers stabilized by di-sulfide bond (under oxidative conditions). Both of these types of FGF modifications fall within the definition of chemical linkages described below.

Other mutations that can be made result in reduced heparin binding, e.g., these mutants have mutations at the heparin-binding sites such that the mutated residues (e.g. K—>A) would not interact with heparin; reduced receptor binding, e.g. these mutants have mutations at the receptor binding site of FGF such that the mutated residues do not interact with FGFR. In some aspects it may also be desirable to modify the FGF monomers to prevent dimerization, e.g. for controls or competitors, or to prevent FGF activity. Dimerization (non-covalent) may be inhibited with e.g. Y124R which is designed to disrupt dimerization by introducing the mutated residue to block the interface between the two proteins.

In the description herein, reference is made to the amino acid residues and residue positions of native FGF2 with 9 N-terminal residues deleted disclosed in SEQ ID NO.: 7. In particular, residues and residue positions are referred to as “corresponding to” a particular residue or residue position of FGF. As will be obvious to one of ordinary skill in the art, these positions are relative and, therefore, insertions or deletions of one or more residues would have the effect of altering the numbering of downstream residues. In particular, N-terminal insertions or deletions would alter the numbering of all subsequent residues. Therefore, as used herein, a residue in a recombinant modified FGF2 dimer will be referred to as “corresponding to” a residue of the full FGF2 if, using standard sequence comparison programs, they would be aligned. Many such sequence alignment programs are now available to one of ordinary skill in the art and their use in sequence comparisons has become standard. As used herein, this convention of referring to the positions of residues of the recombinant modified FGF dimers by their corresponding native FGF residues shall extend not only to embodiments including N-terminal insertions or deletions but also to internal insertions or deletions.

In addition, in the description herein, certain substitutions of one amino acid residue for another in a recombinant FGF or FGF dimer are referred to as “conservative substitutions.” As used herein, a “conservative amino acid substitution” or “conservative substitution” refers to an amino acid substitution in which the substituted amino acid residue is of similar charge as the replaced residue and is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered “conservative substitutions” even if the residues are in different groups (e.g., replacement of phenylalanine with the smaller isoleucine). The term “conservative amino acid substitution” also refers to the use of amino acid analogs or variants.

Methods for making amino acid substitutions, additions or deletions are well known in the art and are described in detail in the Examples below. The terms “conservative substitution”, “non-conservative substitutions”, “non-polar amino acids”, “polar amino acids”, and “acidic amino acids” are all used consistently with the prior art terminology. Each of these terms is well-known in the art and has been extensively described in numerous publications, including standard biochemistry text books, such as “Biochemistry” by Geoffrey Zubay, Addison-Wesley Publishing Co., 1986 edition, which describes conservative and non-conservative substitutions and properties of amino acids which lead to their definition as polar, non-polar or acidic.

Even when it is difficult to predict the exact effect of a substitution in advance of doing so, one skilled in the art will appreciate that the effect can be evaluated by routine screening assays, preferably the biological assays described herein. Modifications of peptide properties including thermal stability, hydrophobicity, susceptibility to proteolytic degradation or the ability to interact with the receptor are assayed by methods well known to the ordinarily skilled artisan. For additional detailed description of protein chemistry and structure, see Schulz, G. E. et al., Principles of Protein Structure, Springer-Verlag, New York, 1979, and Creighton, T. E., Proteins: Structure and Molecular Principles, W. H. Freeman & Co., San Francisco, 1984.

Additionally, some of the amino acid substitutions are non-conservative substitutions. In certain embodiments where the substitution is remote from the active or binding sites, the non-conservative substitutions are easily tolerated provided that they preserve the tertiary structure characteristic of native FGF, thereby preserving the active and binding sites. Non-conservative substitutions, such as between, rather than within, the above groups (or two other amino acid groups not shown above), which will differ more significantly in their effect on maintaining (a) the structure of the peptide backbone in the area of the substitution (b) the charge or hydrophobicity of the molecule at the target site or (c) the bulk of the side chain.

The proteins of the present invention can also comprise, in addition to the 20 standard amino acids, non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxyethyl-cysteine, hydroxyethylhomocysteine, nitroglutamine, homoglutamine, pipecolic acid, tert-leucine, norvaline, 2-azaphenylalanine, 3-azaphenylalanine, 4-azaphenyl-alanine, 4-fluorophenylalanine, 4-hydroxyproline, 6-N-methyl lysine, 2-aminoisobutyric acid, isovaline and .alpha.-methyl serine.

Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations are carried out in a cell free system comprising an E. coli S30 extract and commercially available enzymes and other reagents. Proteins are purified by chromatography. See, for example, Robertson et al., J Am. Chem. Soc. 113:2722, 1991; Ellman et al., Meth. Enzymol. 202:301, 1991; Chung et al., Science 259:806-09, 1993; and Chung et al., Proc. Natl. Acad. Sci. USA 90:10145-49, 1993. In a second method, translation is carried out in Xenopus oocytes by microinjection of mutated mRNA and chemically aminoacylated suppressor tRNAs (Turcatti et al., J. Biol. Chem. 271:19991-98, 1996). In a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, e.g., Koide et al., Biochem. 33:7470-76, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci 2:395-403, 1993).

Additionally, the linker sequences, and the N/C terminal tags can be substituted with other sequences for defined purposes, such as integrin binding sequences, protease sites (e.g., in the linker to manipulate cleavage), epitopes, etc.

The FGF DNA used in generating the FGF dimers may be natural, recombinant or synthetic. Thus, DNA starting material is isolated from tissue or tissue culture, constructed from oligonucleotides using conventional methods, obtained commercially, or prepared by isolating RNA coding for FGF from fibroblasts, and using this RNA to synthesize single-stranded cDNA which is used as a template to synthesize the corresponding double stranded DNA.

The term “chemical linkage” as used herein refers to a direct linkage between the two monomers. The direct linkage may be covalent or non-covalent. In some preferred embodiments the chemical linkage is a covalent disulfide linkage, arising from the interaction between two cysteine residues that have been incorporated into the monomers. Examples of monomers having cysteines incorporated therein that can produce disulfide bonds include those having sequences set forth in SEQ. ID NOs.: 2-4. Exemplary methods for generating these types of FGF dimers having a chemical linkage is set forth in the Examples section.

In addition to the modified FGF dimers of the invention, some embodiments and aspects of the invention utilize naturally occurring FGF dimers. Preferably the naturally occurring FGF dimers are stabilized. Stabilizing agents include, but are not limited to, glycosaminoglycans, such as heparin, heparin fragments, heparan sulfate and dermatan sulfate, or glucan sulfates, such as dextran sulfate, Tri 3 oligosaccharides, and cyclodextrin sulfate. Stabilized FGF monomers of this type are described, for example, in EP 251 806, EP 267 015, EP 312 208, EP 345 660, EP 406 856, EP 408 146, WO 89-12464, WO 90-01941 and WO 90-03797.

HLGAGs that enhance ‘natural’ dimerization of FGF are in general oligosaccharides of 8-10 monosaccharide units long with 2-O and N-sulfation. The HLGAG can also be obtained from heparin or its fragments. Tri 3 is a unique HLGAG that promotes dimerization in that it is both short (three saccharides long) and undersulfated. It has previously been described in Omitz et al in Science 268:432.

The FGF dimers of the invention have important therapeutic and diagnostic utilities. For instance, the FGF dimers can promote vascularization, cell growth, and/or cell survival, and thus have application in tissue repair such as healing of wounds, burns, bone fractures, surgical abrasions, gastrointestinal ulcers, and the like as well as tissue repair during ischemia and myocardial infarction via neovascularization of ischemic tissue. FGF2 is also effective in maintaining certain hematopoietic lineages in long term primary bone marrow culture and for the survival and possible differentiation of hematopoietic progenitor cells.

The FGF dimers of the invention may be used for any of the same purposes as native FGF. For instance, the FGF dimers can be used to promote angiogenesis. The invention is useful in a variety of in vitro, in vivo and ex vivo methods.

The FGF dimers may be used, for instance, in a method for promoting angiogenesis. In this method an effective amount for promoting angiogenesis of the FGF dimer is administered to a subject in need of treatment thereof. Angiogenesis as used herein is the formation of new blood vessels in tissue in response to stimuli. The methods for promoting angiogenesis are particularly useful in the treatment of ischemic tissues which are deprived of blood perfusion for any reason, such as the result of coronary or peripheral artery disease which deprives the tissue of adequate blood flow. Neovascularization, or angiogenesis, is the growth and development of new arteries. It is critical to the normal development of the vascular system, including injury repair.

Disorders in which angiogenesis is desirable include, for example, various ulcerating diseases of the gastrointestinal tract such as regional ileitis, ulcerative colitis and peptic ulcer (either duodenal or gastric); tissue injuries such as bums, wounds, postoperative tissues, thrombosis, arteriosclerosis; musculo-skeletal conditions such as bone fractures, ligament and tendon repair, tendonitis and bursitis; skin conditions such as minor bums, cuts, lacerations, bed sores; slow-healing and chronic ulcers such as those seen in diabetics; and in tissue repair during ischaemia and myocardial infarction.

Thus the methods of the invention are useful for treating cerebral ischemia. Cerebral ischemia may result in either transient or permanent deficits and the seriousness of the neurological damage in a patient who has experienced cerebral ischemia depends on the intensity and duration of the ischemic event. A transient ischemic attack is one in which the blood flow to the brain is interrupted only briefly and causes temporary neurological deficits, which often are clear in less than 24 hours. Symptoms of TIA include numbness or weakness of face or limbs, loss of the ability to speak clearly and/or to understand the speech of others, a loss of vision or dimness of vision, and a feeling of dizziness. Permanent cerebral ischemic attacks, also called stroke, are caused by a longer interruption in blood flow to the brain resulting from either a thromboembolism. A stroke causes a loss of neurons typically resulting in a neurologic deficit that may improve but that does not entirely resolve. Thromboembolic stroke is due to the occlusion of an extracranial or intracranial blood vessel by a thrombus or embolus. Because it is often difficult to discern whether a stroke is caused by a thrombosis or an embolism, the term “thromboembolism” is used to cover strokes caused by either of these mechanisms.

The methods of the invention in some embodiments are directed to the treatment of acute thromboembolic stroke using FGF dimers. An acute stroke is a medical syndrome involving neurological injury resulting from an ischemic event, which is an interruption in the blood supply to the brain.

An effective amount of an FGF dimer alone or in combination with another therapeutic for the treatment of stroke is that amount sufficient to reduce in vivo brain injury resulting from the stroke. A reduction of brain injury is any prevention of injury to the brain which otherwise would have occurred in a subject experiencing a thromboembolic stroke absent the treatment of the invention. Several physiological parameters may be used to assess reduction of brain injury, including smaller infarct size, improved regional cerebral blood flow, and decreased intracranial pressure, for example, as compared to pretreatment patient parameters, untreated stroke patients or stroke patients treated with thrombolytic agents alone.

The FGF dimers may be used alone or in combination with a therapeutic agent for treating stroke. Examples of therapeutics useful in the treatment of stroke include anticoagulation agents, antiplatelet agents, and thrombolytic agents.

Anticoagulation agents prevent the coagulation of blood components and thus prevent clot formation. Anticoagulants include, but are not limited to, heparin, warfarin, coumadin, dicumarol, phenprocoumon, acenocoumarol, ethyl biscoumacetate, and indandione derivatives.

Antiplatelet agents inhibit platelet aggregation and are often used to prevent thromboembolic stroke in patients who have experienced a transient ischemic attack or stroke. Antiplatelet agents include, but are not limited to, aspirin, thienopyridine derivatives such as ticlopodine and clopidogrel, dipyridamole and sulfinpyrazone, as well as RGD mimetics and also antithrombin agents such as, but not limited to, hirudin.

Thrombolytic agents lyse clots which cause the thromboembolic stroke. Thrombolytic agents that have been used in the treatment of acute venous thromboembolism and pulmonary emboli and are well known in the art (e.g. see Hennekens et al, J Am Coll Cardiol; v. 25 (7 supp), p. 18S-22S (1995); Holmes, et al, J Am Coil Cardiol; v.25 (7 suppl), p. 10S-17S(1995)). Thrombolytic agents include, but are not limited to, plasminogen, a₂-antiplasmin, streptokinase, antistreplase, tissue plasminogen activator (tPA), and urokinase. “tPA” as used herein includes native tPA and recombinant tPA, as well as modified forms of tPA that retain the enzymatic or fibrinolytic activities of native tPA. The enzymatic activity of tPA can be measured by assessing the ability of the molecule to convert plasminogen to plasmin. The fibrinolytic activity of tPA may be determined by any in vitro clot lysis activity known in the art, such as the purified clot lysis assay described by Carlson, et. al., Anal. Biochem. 168, 428-435 (1988) and its modified form.

The FGF dimers are also useful for treating and preventing neurodegenerative disease and for promoting nerve regeneration and spinal chord repair. FGF is involved in regulating dopaminergic neuron survival and metabolism, either directly, or indirectly by effecting adjacent cells (Dal Toso et al. J Neurosci., 8(3): 733-745 (1988)). The degeneration of the substantia nigra dopaminergic neurons which characterizes Parkinson's Disease is normally treated using pharmacological interventions to augment the declining natural dopamine supply to the striatum. Neuronal grafts, using embryonic substantia nigral tissue also have shown some potential for relieving experimentally induced Parkinsonism in rodents and primates and in some human Parkinsonian patients. The FGF dimers may be used to treat neural cells to produce differentiating or differentiated dopaminergic cells prior to transplant of the dopaminergic cells into the patient. The term “dopaminergic neural tissue” refers to the tissue from regions of the CNS that are known, in the mature state, to contain significant numbers of dopaminergic cell bodies.

Purified populations of differentiated dopaminergic cells, derived from primary culture or from the proliferated precursor progeny of neural stem cells, may be implanted into dopamine deficient regions of the brain of a recipient. Alternatively, cells that have been cultured in a culture medium that induces the formation of dopaminergic cells may be implanted into the brain prior to the completion of the differentiation process. Following implantation, the differentiation of dopaminergic cells may be completed in vivo. Any suitable method for purifying the cells may be used, or the cells could be implanted together with other neural cells. Any suitable method for the implantation of dopaminergic cells or precursor cells near the region of dopamine depletion may be used. Methods taught in U.S. Pat. No. 5,082,670 to Gage et al. for the injection of cell suspensions, such as fibroblasts, into the CNS may be employed for the injection of the differentiated dopaminergic cells prepared using the FGF dimers. Additional approaches and methods may be found in Neural Grafting in the Mammalian CNS, Bjorklund and Stenevi, eds., (1987). Xeno and/or allografts may require the application of immunosuppressive techniques or induction of host tolerance to enhance the survival of the implanted cells.

The FGF dimers may be used in treatment of disorders associated with myocardial infarction, congestive heart failure, hypertrophic cardiomyopathy and dilated cardiomyopathy. FGF dimers of the present invention may also be useful for limiting infarct size following a heart attack, for promoting angiogenesis and wound healing following angioplasty or endarterectomy, for developing coronary collateral circulation, for revascularization in the eye, for complications related to poor circulation such as diabetic foot ulcers, for stroke (as described above), following coronary reperfusion using pharmacologic methods and other indications where angiogenesis is of benefit. FGF dimers may be useful for improving cardiac function, either by inducing cardiac myocyte neogenesis and/or hyperplasia, by inducing coronary collateral formation, or by inducing remodeling of necrotic myocardial area.

Additionally a role for FGF in osteogenesis has recently been reported in individual cases, for example in Biomaterials 11, 38-40 (1990). It is reported in Acta Orthop. Scand. 60, (4) 473-476 (1989) that an increased content of mineralized tissue was found in implants of demineralized bone matrix (DBM) which had been charged with recombinant human FGF and implanted intramuscularly into rats. Thus the FGF dimers also find use in bone remodeling and repair. Bone remodeling is the dynamic process by which tissue mass and skeletal architecture are maintained. The process is a balance between bone resorption and bone formation, with two cell types thought to be the major players. These cells are the osteoblast and osteoclast. Osteoblasts synthesize and deposit matrix to become new bone.

The FGF dimers may also be useful for the treatment of nervous system diseases. A nervous system disease is a disease involving one or more nerve cells, which may be a disease of the central nervous system or of the peripheral nervous system. Diseases or disorders of the central nervous system include but are not limited to Pathophysiologic complications such as herniations and cerebral edema; Malformations and developmental diseases such as neural tube defects and syringomyelia and hydromyelia; Perinatal brain injury such as cerebral palsy; Trauma such as parenchymal injuries (concussion, etc.), traumatic vascular injury (e.g., hematoma and traumatic subarachnoid hemorrhage and traumatic intraparenchymal hematoma), and spinal cord injury; Cerebrovascular Disease such as hypoxia, ischemia and infarction, nontraumatic intracranial hemorrhage, vascular malformations, hypertensive cerebrovascular disease; Infections such as meningitis, chronic meningoencephalitis (e.g., tuberculous and chronic meningitis, neurosyphilis, lyme disease), viral encephalitis, spongiform encephalopathies, fungal infection; Demyelinating diseases such as multiple sclerosis and acute disseminated encephalomyelitis; Degenerative diseases such as Alzheimer's disease, Pick's disease, Parkinsonism, Huntington's disease, Friedreich's ataxia; Genetic diseases of metabolism (affects CNS); Toxic and acquired metabolic diseases such as vitamin deficiencies; and Neurocutaneous syndromes such as neurofibromatosis (NF1, NF2), tuberous sclerosis and Von Hippel-Lindau disease.

Diseases or disorders of the peripheral nervous system include but are not limited to Inflammatory neuropathy such as Guillain-Barre syndrome and chronic inflammatory demyelinating polyradiculoneuropathy; Infectious neuropathy such as leprosy, diptheric neuropathy, and varicella-zoster virus (can also affect CNS); Hereditary neuropathy such as hereditary motor and sensory neuropathy I (HMSN I), HMSN II, HMSN III, hereditary sensory and autonomic neuropathy I (HSAN I), HSAN II, HSAN III, adrenoleukodystrophy, familial amyloid polyneuropathies, porphyria, Refsum's disease; and Acquired metabolic and toxic neuropathies such as peripheral neuropathy induce by adult-onset diabetes mellitus, from metabolic and nutritional causes, toxic causes or induced by trauma.

The FGF dimers are also useful for any other indication that FGF is otherwise useful for. Since the compositions have a mechanism of action similar to native FGF, but with a higher efficacy, these compounds are useful for any of the same uses as native FGF. These include the diseases described above as well as any other indications that FGF is useful for.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants, and optionally other therapeutic ingredients.

In some aspects the compositions are formulated for delivery to a subject. A composition is formulated for delivery to a subject if it is in a material that is non-toxic to the subject. For instance, a material that is formulated in an SDS buffer is not formulated for delivery to a subject. In some embodiments the FGF dimers are also included in delivery vehicles that promote more efficient or sustained release delivery. These vehicles are described in more detail below.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, which comprise FGF dimers together with one or more pharmaceutically acceptable carriers and optionally other therapeutic ingredients. In some embodiments the pharmaceutical compositions are formulated for in vivo delivery. A preferred mode of delivery includes the use of sustained release carriers. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with the FGF dimers, and with each other, in a manner such that there is no interaction which would substantially impair the desired pharmaceutical efficiency.

A variety of administration routes are available. The particular mode selected will depend, of course, upon the particular FGF dimer selected, the particular condition being treated and the dosage required for therapeutic efficacy. The methods of this invention, generally speaking, may be practiced using any mode of administration that is medically acceptable, meaning any mode that produces effective levels of FGF activity without causing clinically unacceptable adverse effects. A preferred mode of administration is a parenteral route. The term “parenteral” includes subcutaneous injections, intravenous, intramuscular, intraperitoneal, intra sternal injection or infusion techniques. Other modes of administration include oral, mucosal, rectal, vaginal, sublingual, intranasal, intratracheal, inhalation, ocular, transdermal, etc.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990.

The compositions may conveniently be presented in unit dosage form and may be prepared by any of the methods well known in the art of pharmacy. All methods include the step of bringing the active FGF dimers into association with a carrier which constitutes one or more accessory ingredients. In general, the compositions are prepared by uniformly and intimately bringing the polymer into association with a liquid carrier, a finely divided solid carrier, or both, and then, if necessary, shaping the product. The polymer may be stored lyophilized.

Other delivery systems can include time-release, delayed release or sustained release delivery systems. Such systems can avoid repeated administrations of the FGF dimers of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775 (Kent); 4,667,014 (Nestor et al.); and 4,748,034 and 5,239,660 (Leonard) and (b) diffusional systems in which an active component permeates at a controlled rate through a polymer, found in U.S. Pat. Nos. 3,832,253 (Higuchi et al.) and 3,854,480 (Zaffaroni). In addition, a pump-based hardware delivery system can be used, some of which are adapted for implantation.

A subject is any human or non-human vertebrate, e.g., dog, cat, horse, cow, pig, goat, rabbit, mouse, rat.

The invention also encompasses screening assays. One screening assay of the invention is useful for identifying an FGF dimer binding compound. The assay involves contacting a library of compounds with the FGF dimer of the invention and identifying a compound that binds the FGF dimer to identify the FGF dimer binding compound. The assay may optionally include the further step of determining whether the FGF binding compound is an FGF inhibitor by determining whether the FGF binding compound can block FGF dimer interaction with an FGF receptor. These types of assays are routine in the art. One of skill in the art is now enabled to perform these assays based on the teachings disclosed in the instant invention. Thus, the FGF dimers can be used to screen libraries of compounds, such as small molecules or peptide libraries. The invention also includes compositions of the molecules identified in these assays, e.g., the FGF dimer binding compound and the FGF inhibitor. The FGF inhibitor can be used for inhibiting FGF activity in a subject by administering to the subject. These inhibitor compounds are particularly useful for inhibiting angiogenesis and thus are potent anti-cancer agents. The inhibitors are also useful for treating chronic inflammation.

The following examples are provided to illustrate specific instances of the practice of the present invention and are not to be construed as limiting the present invention to these examples. As will be apparent to one of ordinary skill in the art, the present invention will find application in a variety of compositions and methods.

### Examples

Materials—Ampicillin, isopropyl β-D-thiogalactopyranoside (IPTG), 1,10-phenthroline, sodium chlorate and dithiothreitol (DTT) were from Sigma (St. Louis, Mo.). Recombinant human wild-type FGF2 was a gift from Scios Nova (Mountain View, Calif.). The expression vector pET14b variant was a generous gift from D. Ornitz of Washington University. Heparin sodium USP porcine intestinal mucosa was from Kabi Pharmacia (Franklin, Ohio). Ready-Gel (15% polyacrylamide gel), Bradford assay kit, immunoblot assay kit and silver staining kit were from Bio-Rad (Hercules, Calif.).

Site-directed Mutagenesis, Protein Expression and Purification of Cysteine Mutant—Site-directed mutagenesis was carried out through a two-step PCR procedure as described previously (Higuchi, R. (1990) PCR Protocols: A guide to Methods and Applications (Innis, M. A. et al. Eds), Academic Press, San Diego). PCR products were subcloned into pCR2.1-TOPO vector (Invitrogen, Carlsbad, Calif.). Inserts were subcloned into a variant of pET14b expression vector through the NdeI/SpeI sites. To express recombinant protein, overnight culture of BL21 cells was transferred to two 500 ml LB medium supplemented with ampicillin (400 mg/L) and allowed to grow with shaking at 37° C. until cell density reached OD₆₀₀ of ˜0.5. IPTG (1 mM) was added to induce protein expression for 2 h. Protein purification by Ni-chromatography was performed as previously described (Ernst, S. et al. (1996) Biochem J 315(Pt 2), 589-97; Padera, R. et al. (1999) Faseb J 13(13), 1677-87). Purity of the protein was assessed by SDS-PAGE under non-reducing conditions and concentration was determined by Bradford assay using recombinant wild-type FGF2 as control.

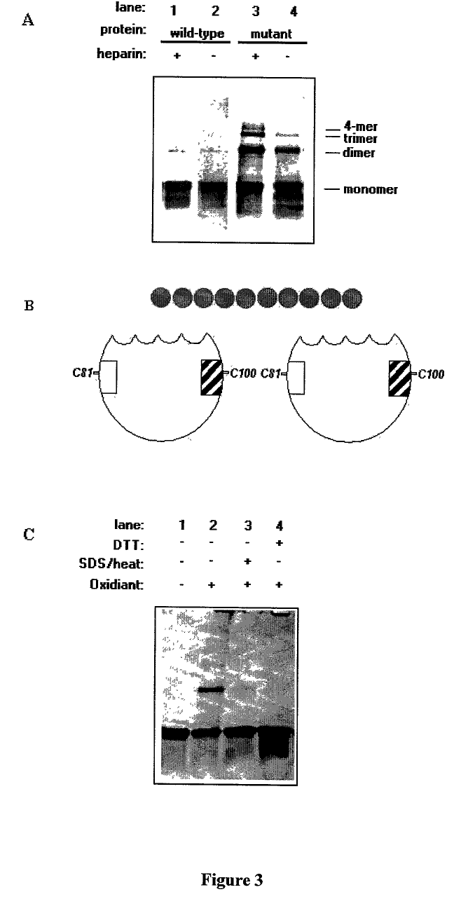

Oxidative Crosslinking—Purified protein was buffer-exchanged into HEPES buffer with 10 kDa molecular mass cut-off membranes (Millipore, Beverly, Mass.). Oxidative crosslinking was performed by incubating 50 μg protein (30 μM final) with 750 μM Cu²⁺-phenanthroline (made from a 1:1 mixture of 25 mM CuSO₄ and 130 mM phenanthroline) in 100 μl reaction volume at room temperature for 10 min. Longer incubation time (up to 2 h) did not significantly increase the amount of oligomer formed. For heparin treatment, protein was incubated with 3 μM heparin for 1 h prior to crosslinking. The protein to heparin ratio was 10:1, which was previously shown to be optimal for FGF2 dimer formation (Davis, J. et al. (1999) Biochem J341 (Pt 3), 613-20). Other reaction conditions are indicated in the legend to FIG. 3. The reaction was terminated with 0.1 M EDTA and 10 mM iodoacetic acid. Crosslinked products were analyzed by electrophoresis in 15% non-reducing SDS-PAGE gels followed by silver staining.

Conformational Studies—Conformational studies were performed with the Insight II package (Molecular Simulations, Burlington, Mass.) on a Silicon Graphics workstation (Mountain View, Calif.). The coordinates of FGF2 dimer in the FGF2-FGFR1 crystal structure (entry: 1CVS) and that of free FGF2 (entry: 4FGF) were obtained from the Protein Data Bank. The sequential dimer was constructed from 4FGF by translating the coordinates along the 31 Å axis.

The linker used in the experiment contained a tripeptide with the sequence of GAL. However, since the N- and C-termini of FGF2 in most of the crystal structures are disordered, the modeled linker included the tripeptide sequence and the disordered residues of FGF2. The sequence of the linker was of the form Cₜₑᵣₘ-GAL-Nₜₑᵣₘ, where Cₜₑᵣₘ and Nₜₑᵣₘ are the disordered C- and N-termini of FGF2, respectively. By deleting residues from the disordered N-terminus, linkers of different lengths could be obtained. The most optimal structure for each of the linkers was obtained as follows. Combinations of structures for the linker were generated from the C-terminus of one of the FGF2 monomers to the N-terminus of the other monomer in both the receptor-bound and sequential dimer using the homology modeling of Insight II. A good starting structure from the randomly generated linker structures in each FGF2 dimer was subject to energy minimization with the Newton-Raphson method until convergence. Potentials were assigned using the Consistent Valence forcefield. Interchanging the N- and C-termini among monomers did not lead to significant changes in the model of the crosslinked dimer, and therefore it did not affect the interpretation of the results.

Construction of dimeric or dFGF2—Based on the results from the conformational studies, the two DNA sequences of FGF2 were ligated and subcloned into an expression vector as outlined in FIG. 4(A). NdeI/SacI sites were introduced by PCR to the 5′ and 3′ ends of the first sequence while SacI/SpeI sites were introduced to the second. Both the first and the second sequences encode for the FGF2 with the first 9 N-terminal residues removed. To facilitate purification of dFGF2, a 6× His tag and a thrombin cleavage site were introduced by PCR to the 5′ end of the first sequence, and a T7 tag and another thrombin cleavage site were introduced similarly to the 3′ end of the second sequence. Upon subcloning of the PCR product of the first sequence into pCR2.1-TOPO (which carries an internal SpeI site), a SacI/SpeI double digest was performed to linearize the vector. The PCR product from the second sequence was subcloned similarly and the insert was excised by a SacI/SpeI double digest. Ligation between the linearized vector and the insert from the second sequence resulted in a fused DNA of two tandemly-linked FGF2 DNA sequences. DNA sequencing was performed to confirm the identities of the fused DNA sequences. Protein expression and purification were performed as above except that a T7-affinity column was used as described by the manufacturer (Novagen, Madison, Wis.) after Ni-chromatography. Biochemical studies were performed to ensure that dFGF2 was folded properly. Immunoblot analysis using a monoclonal antibody against the native form of wild-type FGF2 showed that the elutants from Ni and T7-affinity chromatographies were recognized by the antibody in a concentration dependent fashion.

CD spectroscopy—dFGF2 was concentrated to 1 μM and buffer-exchanged into 10 mM sodium phosphate, pH 7.2. CD spectroscopy of dFGF2 was performed in a quartz cell with a 1 mm pathlength (Starnz, Atascadero, Calif.) at room temperature. Data were recorded in an average of 20 scans between 195 nm and 260 nm on an Aviv 62SD spectropolarimeter.

Protein Mass Spectrometry—MALDI-MS was completed by diluting a solution of FGF2, FGFR1, and an HLGAG decasaccharide to 20 μM in 10 mM sodium phosphate pH 7.0. To 1 μL of this sample was added an equimolar amount of the dFGF2 from which the 6×His tag and the T7 tag had been removed by thrombin cleavage as described by the manufacturer (Novagen, Madison, Wis.). The sample was allowed to come to equilibrium for 30 minutes at 4° C. 1 μL of the sample was then immediately spotted on the MALDI target with 1 μL of a saturated sinapinic acid solution in 50% acetonitrile. After drying, the sample was washed with water, dried under a stream of nitrogen, and subjected to mass spectral analysis. MALDI-MS spectra were acquired in the linear mode using a Voyager Elite reflectron time-of-flight instrument (PerSeptive Biosystems, Framingham, Mass.) fitted with a 337-nm nitrogen laser. Delayed extraction was used to increase resolution (25 kV, grid at 91%, guide wire at 0.25%, pulse delay 350 ns, low mass gate at 2000). As indicated in the text, all species were within 0.1% of their theoretical values.

SMC Proliferation Assay—Smooth muscle cells (SMC) isolated from bovine aorta were maintained in propagation media supplemented with 10% bovine calf serum (BCS), 2 mM L-glutamine and antibiotics. Proliferation assay of SMC, as measured by tritium incorporation, was performed as follows. Cells were split at 95% confluence and seeded onto 24 well plates at 1 ml per well. After 24 h, cells were serum-starved in media supplemented with 0.1% BCS for another 24 h. An appropriate amount of growth factors was added to 8 wells for each protein concentration tested. 75 mM sodium chlorate was added to half of the wells for each condition. After 21 h, [³H] thymidine (1 μCi/ml) was applied to each well and incubated for 3 h. Cells were washed with PBS and 0.5 ml 1M NaOH was subsequently added. The contents of each well were transferred to scintillation vials filled with 5 ml ScintiSafe Plus 50% (Fischer, N.J.) scintillation fluid. Total [³H] thymidine incorporation was measured by liquid scintillation counting.

HUVEC Survival Assay—Human umbilical vein endothelial cells (HUVEC) in passage three or four were cultured on 1% gelatin-coated tissue culture plates in medium M199 (BioWhittaker, Walkerville, Md.) supplemented with 20% fetal bovine serum (FBS). After 24 h, HUVEC were trypsinized briefly at 37° C., washed twice with PBS and resuspended in medium containing 0.5% FBS and 1% bovine serum albumin (BSA). The cells were seeded at a density of approximately 1-2×10⁴ per well onto 96-well plates coated with fibronectin-like polymer (Sigma, St Louis, Mo.). Appropriate amounts of growth factors were added to the wells using a multi-channel pipette. Each experimental condition was tested in six different wells. Cell viability was assessed after 18 h using a colorimetric MTS assay (Promega, Madison, Wis.) by measuring absorbance at 490 nm.

Angiogenic Assay in the Rat Cornea—Pellets containing sucralfate with FGF2 or sucralfate alone were prepared as described by Kenyon et al (Kenyon, B. M. et al. (1996) Inves Ophthalmol Vis Sci 37(8), 1625-32). Briefly, suspensions of sterile FGF2 solution containing appropriate amounts of mFGF2 (5 μg and 20 μg) and dFGF2 (5 μg) were prepared and speed vacuumed for 5 min. 10 μl of 12% Hydron in ethanol was added and the suspension was deposited onto an autoclave sterilized nylon mesh. The mesh was stacked between two layers of fiber covered with a thin film of Hydron. After drying on a sterile petri dish for 30 min, the fibers of the mesh were pulled apart under a microscope. With the aid of a dissecting microscope, uniformly sized pellets were selected from approximately 200 pellets produced. Each pellet contained approximately 1.5 pmole and 6 pmole mFGF2 or 0.7 pmole dFGF2. Control pellets containing no FGF2 were also prepared.

For pellet implantation, Sprague Dawley rats (male, 400-450 g, n=5) were anesthetized with Ketamine (80 mg/kg) or Xylazine (10 mg/kg). Using an operating microscope, an intrastromal linear keratomy was performed with a surgical blade (Bard-Parker no. 15, Becton Dickenson, Franklin Lakes, N.Y.) parallel to and 2 mm away from the limbus. A lamellar micropocket was dissected toward the limbus. A single pellet was placed to the base of the pocket with jeweler's forceps. On day 6 after the implantation, the corneal angiogenesis was photographed with a slit lamp and the area of angiogenesis assessed as described (Kenyon, B. M. et al. (1996) Inves Ophthalmol Vis Sci 37(8), 1625-32).

### Results

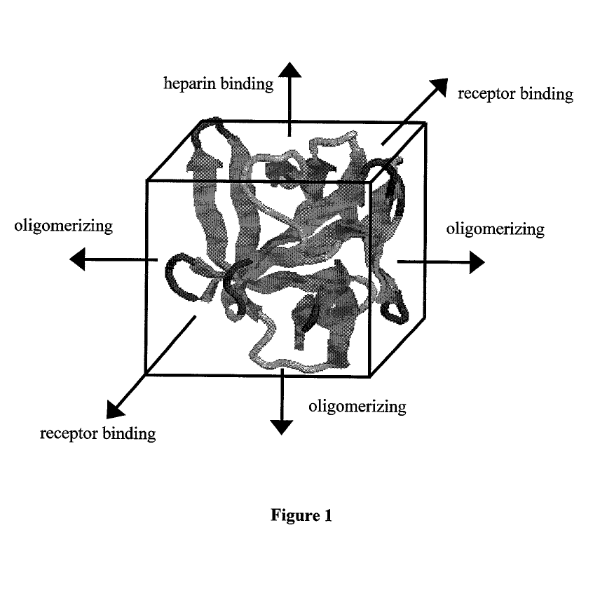

Framework for the present study—The three dimensional structure of FGF2 has been thoroughly elucidated by a variety of biophysical techniques, including solution NMR and crystallography (Faham, S. et al. (1996) Science 271(5252), 1116-20; Moy, F. J. et al. (1997) Biochemistry 36(16), 4782-91; DiGabriele, A. D. et al. ((1998) Nature 393(6687), 812-7; Plotnikov, A. N. et al. (1999) Cell 98(5) 641-50; Plotnikov, A. N. et al. (2000) Cell 101(4), 413-24; Stauber, D. J. et al. (2000) Proc Natl Acad Sci USA 97(1), 49-54; Schlessinger, J. et al. (2000) Mol Cell 6(3), 743-50; Moy, F. J. et al. (1996) Biochemistry 35(42), 13552-61; Zhang, J. D. et al. (1991) Proc Natl Acad Sci USA 88(8), 3446-50; Eriksson, A. E. et al. (1993) Protein Sci 2(8), 1274-84). All have pointed to roughly the same basic structure for FGF2, whether free, bound to its HLGAG ligand, or complexed with the receptor. An analysis of all of these structures, suggests that three orthogonal surfaces exist on FGF2 (FIG. 1). As indicated in the figure, the first surface has been implicated in binding of FGF2 to its high affinity protein receptor. Through rigorous biochemical and site-directed mutagenesis studies, a second, orthogonal surface has been implicated in HLGAG binding. The third surface, orthogonal to both of the first two has been implicated in FGF2 oligomerization.

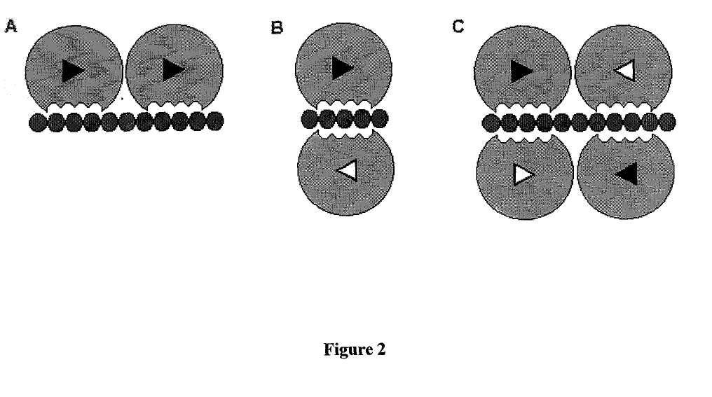

Within the third surface, biochemical and structural studies have suggested different modes of FGF2 oligomerization both in the presence and absence of HLGAGs (Venkataraman, G. et al. (1996) Proc Natl Acad Sci USA 93(2), 845-50; Moy, F. J. et al. (1997) Biochemistry 36(16), 4782-91; DiGabriele, A. D. et al. (1998) Nature 393(6687), 812-7). As schematically represented in FIG. 2, three modes of HLGAG-induced FGF2 dimerization are possible. Specific protein-protein contacts are involved in both the sequential and symmetrical FGF2 dimers (FIG. 2(A) and (C), respectively) but not in the HLGAG-bridged or sandwich dimer (FIG. 2(B)). Earlier it was demonstrated that FGF2 was capable of dimerization and oligomerization in the absence of heparin using an amine-specific chemical crosslinker with an 11 Å spacer (Davis, J. C. et al. (1999) Biochem J 341 (Pt 3), 613-20). This composition, however, was never purified and was not tested for biological activity. The dimeric composition was manipulated in biochemical assays and was only formulated in toxic materials that are not pharmaceutically acceptable. This observation was not consistent with the proposed HLGAG-bridged dimer in FIG. 2(B) since, in this FGF sandwich model, there are no residues on neighboring FGF2 molecules proximate to one another and thus available for covalent crosslinking with an 11 Å spacer (additional experiments described below also were not consistent with this dimer mode). Therefore, we focused our initial experiments on determining whether either of the dimer models involving protein contacts (represented in FIG. 2(A) and (C)) are accurate representations of FGF2 dimerization mediated by HLGAGs.

Strategy to investigate FGF dimerization: Oxidative crosslinking through surface exposed cysteine residues on FGF2—To establish the presence of proximal contacts between FGF2 molecules and distinguish between different modes of FGF2 dimerization, we performed oxidative crosslinking experiments targeting the cysteine residues of FGF2 using copper phenanthroline, an oxidative agent used widely for disulfide bond formation (Bisaccia, F. et al. (1996) Biochem Biophys Acta 1292(2) 281-88. This approach is anticipated to probe for atomic distance interactions between the FGF molecules, through the introduction of a disulfide bond between two FGF2 molecules. As discussed below, by taking advantage of the surface exposed cysteine residues in FGF2 and through rationally engineering cysteine residues on the surface of FGF2, we systematically explored possible modes of FGF2 dimerization.

Oxidative crosslinking of wild-type FGF2—There are four cysteines in FGF2, two of which are surface exposed (C69 and C87) and two of which are buried in the protein core (C25 and C92). The surface positions of the two exposed cysteines (C69 and C87) in wild-type FGF2 are related to each other by 90 degrees. Taking advantage of the surface exposed cysteine residues in the wild-type structure of FGF2, we performed oxidative crosslinking studies to test the proposed symmetrical mode of FGF2 dimerization of FIG. 2(C), as this model predicts facile crosslinking between two FGF2 molecules. Under mild oxidative conditions, wild-type FGF2 showed very little oligomer formation in the presence and absence of heparin (FIG. 3(A), lane 1 and 2, several control experiments were performed to ensure authenticity of the data, and are described below). The absence of significant dimers or oligomers suggests that either the FGF-FGF interface does not involve molecular contacts or that the contacts are such that the two surface exposed cysteines are not at the dimer interface. Our observation is not consistent with the proposed symmetrical mode of FGF2 dimerization wherein dimerization is mediated by disulfide bond formation between C69 of each monomer (Moy, F. J. et al. (1997) Biochemistry 36(16), 4782-91).

Rational design of the cysteine mutant—In a previous study, we had performed an extensive analysis on all FGF2 crystal structures available at that time, and identified protein-protein interfaces (p-p′ and q-q′) that were conserved along the two unit cell axes (Venkataraman, G. et al. (1996) Proc Natl Acad Sci USA 93(2), 845-50). Based on our analysis, we had proposed a FGF2 dimerization model in which FGF2 molecules are preferentially self-associated in a sequential fashion and HLGAG binding stabilized FGF2 dimers and oligomers that were subsequently presented to FGFR for signaling. In this model, non-covalent FGF-FGF interactions translated along the oligomerization direction (FIG. 2(B)) are expected to lead to FGF2 oligomerization. If this model indeed describes a mode of FGF oligomerization, then we would predict that by substituting cysteine residues near the protein-protein interface between adjacent FGF2 molecules, intermolecular disulfide bonds could be created under mild oxidative conditions. The sequential dimer formed in this fashion would be stabilized by significant protein-protein contacts. As a first step towards testing this hypothesis, we searched for candidate pairs of residues in the p-p′ interface that when mutated to cysteine residues, a disulfide linkage could be generated in a facile manner upon oxidative crosslinking. Through conformational studies, we found that optimal disulfide bond formation would be achieved when R81 and S100 were mutated into cysteines, as schematically represented in FIG. 3(B). The two introduced cysteines are located on the opposite sides of FGF2 such that intramolecular disulfide bond formation would be disfavored. The two original cysteines, C69 and C87, were mutated to serines such that the total number of surface cysteines within the primary amino acid sequence of FGF2 remained the same. This protein, with four mutations (R81C/S100C/C69S/C87S), is hereafter referred to as the cysteine mutant. The cysteine mutant was constructed by site-directed mutagenesis as described under Experimental Procedures. The protein retained biological activity to stimulate cell proliferation as compared to wild-type, suggesting that the introduced mutations did not grossly alter protein folding.

Oxidative crosslinking of the cysteine mutant—Under exactly the same oxidative conditions as applied to wild-type, the cysteine mutant yielded a markedly higher amount of oligomers as compared to wild type FGF2. Notably, the extent of oligomerization was elevated by pre-incubating the protein with heparin (FIG. 3(A), lanes 3 and 4). In addition, crosslinking of a mutant FGF that lacked one of these cysteines at the interface (i.e., either the R81C or S100C mutation) resulted in significantly less oligomer formation, further suggesting that the covalent dimer was formed through disulfide bond formation between the designed C81 and C100. Together, these observations strongly support the sequential mode of FGF2 dimerization and also suggest that the extent and stability of FGF2 oligomers are increased by binding to HLGAGs (Venkataraman, G. et al. (1996) Proc Natl Acad Sci USA 93(2), 845-50). Several controls were performed to ensure the authenticity of specific cysteine-mediated FGF2 oligomerization. Addition of a reducing agent such as DTT converted the observed dimers and oligomers into monomers (FIG. 3(C), lane 4), indicating the original crosslinking pattern was the result of disulfide-linked oligomers. Also, oligomerization was abolished when the cysteine mutant was denatured prior to crosslinking (FIG. 3(C), lane 3), suggesting that oligomerization was mediated through the native structure of the protein and the observed oligomers were not formed due to non-specific protein aggregation. In addition, since two cysteines (C25 and C92) were buried in the protein core, they could potentially contribute to the observed oligomerization if the protein was unfolded during crosslinking. To exclude this possibility, the primary amino acid sequence of the cysteine mutant was further altered by substituting the two internal cysteines with serines (i.e., additional C25S/C92S mutations were introduced). The introduction of these two additional mutations did not change the crosslinking pattern, further indicating that only the surface exposed C81 and C100 contributed to disulfide-induced oligomerization. Taken together, these oxidative crosslinking studies support a model wherein FGF2 monomers form sequential dimers via a substantial protein-protein interface and this interaction is further promoted by binding to HLGAGs. These results are consistent with other experimental studies including analytical ultracentrifugation of FGF2 with an octasaccharide, chemical crosslinking and mass spectrometry of FGF2 with or without the addition of exogenous HLGAGs (Ornitz, D. M. et al. (1992) Mol Cell Biol 12(1) 240-7; Herr, A. B. et al. (1997) J Biol Chem 272(26), 16382-9; Davis, J. C. et al. (1999) Biochem J 341 (Pt 3), 613-20; Venkataraman, G. et al. (1999) Proc Natl Acad Sci USA 96(5), 1892-7).

The crosslinked dimers proved to be difficult to purify for further biochemical and biological characterizations. Therefore, we adopted an alternative strategy of constructing an FGF2 dimer using a combination of conformational studies and genetic engineering tools, enabling us to investigate the biological importance of FGF2 dimers. This latter point is of special importance since the above biochemical studies indicate that while a cis FGF dimer does preferentially form in solution it might only form under non-physiological conditions (i.e., high protein concentrations, heparin:protein ratios of 1:10, etc.). However, by constructing a defined FGF2 dimer and testing its biological activity, we can determine whether the oligomer mode indicated by the biochemical studies, viz., a cis dimer involving substantial protein contact, is able to form an active signaling complex at the cell surface.

Engineering of a tandemly linked FGF2 dimer through a linker to probe contact and non-contact FGF-FGF interactions: Design of a dimeric FGF2—Conformational studies of FGF-FGFR interactions led to the proposal that receptor clustering is facilitated by receptor binding to a FGF dimer (Venkataraman, G. et al. (1999) Proc Natl Acad Sci USA 96(7), 3658-63). However, the recently solved structures of 2:2 FGF-FGFR complexes, which are proposed to be active signaling complexes, revealed no contact between the two FGF molecules (Plotnikov, A. N. et al. (1999) Cell 98(5), 641-50; Plotnikov, A. N. et al. (2000) Cell 101(4), 413-24; Stauber, D.J. et al. (2000) Proc Natl Acad Sci USA 97(1), 49-54; Schlessinger, J. et al. (2000) Mol Cell 6(3), 743-50; Pellegrini, L. et al. (2000) Nature 407(6807), 1029-34).

To determine whether FGF-FGF interaction is important for FGFR binding and concomitant signaling, we “forced” FGF2 molecules into a cis dimerization mode by engineering a dimeric FGF2 protein containing a tripeptide linker. By deleting residues from the N-terminus of the protein we could control the size of the linker between the two FGFs. Since there are at least 15 N-terminal residues that are disordered in all the FGF2 crystal structures including the proposed active FGF2-FGFR crystal structures, we expected that these deletions would not significantly affect the folding of the protein. To find the optimal linker sequence length that would facilitate the distinction between the two modes of FGF-FGF interaction we explored combinations of linker sequences with different lengths that could link the FGF2 monomers in both the FGF-FGF interaction modes as outlined in methods section. Our conformational studies showed a linker with 9 residues deleted from N-terminus would optimally link two FGF2 molecules in the sequential dimer, but would form a highly constrained structure when linking the two FGF2 molecules observed in the FGF2-FGFR1 crystal structure. A dimeric protein (referred to as dFGF2) containing a tripeptide linker and two FGF2s, linked C to N, each with the nine N-terminal residues removed was constructed (FIG. 4). This engineered dFGF2 dimer is an ideal candidate to discriminate between a contacting FGF2 dimer and the non-contacting FGF2 dimer as observed in the FGF2-FGFR1 structure. The protein was expressed in E Coli and purified by two chromatographic steps as described in the Experimental Procedures section.

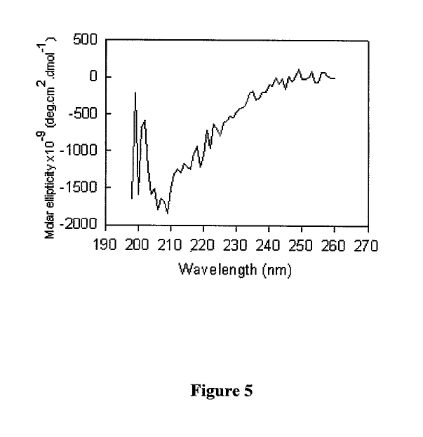

Prior to investigating the biological activity of dFGF2, we performed biochemical studies to ensure that dFGF2 was folded properly. First, as mentioned in the Experimental Procedure section, we assessed the overall folding of the protein by immunoblot. The dFGF construct stained at approximately twice the level of wild-type FGF. In addition, when the purified protein was heat-denatured in the presence of 1% SDS, the intensity in immunoblot was drastically reduced to background level. The above results suggested that dFGF2 was properly folded with respect to the epitope recognized by this antibody. To assess the overall secondary structure, the banding positions of near UV circular dichroic (CD) spectroscopy of dFGF2 was analyzed. The CD spectrum showed a negative minima near 200 nm (FIG. 5), which is characteristic of the native monomeric FGF2 (mFGF2) (Davis, J. C. et al. (1999) Biochem J 341 (Pt 3), 613-20). In addition, dFGF2 bound to a heparin-POROS column and was eluted only at 1.8 M NaCl (compared to 1.2 M NaCl for mFGF2). Not only did this latter result suggest that dFGF2 was properly folded, it also suggested that dFGF2 has a higher affinity for HLGAGs than does mFGF2, perhaps through a cooperative binding interaction between the two linked FGF units and the heparin column. If this is the case, then dFGF2 might have a reduced dependence on exogenous HLGAGs for activity. We explore below the functional attributes, including the effect of HLGAGs on dFGF2 activity.

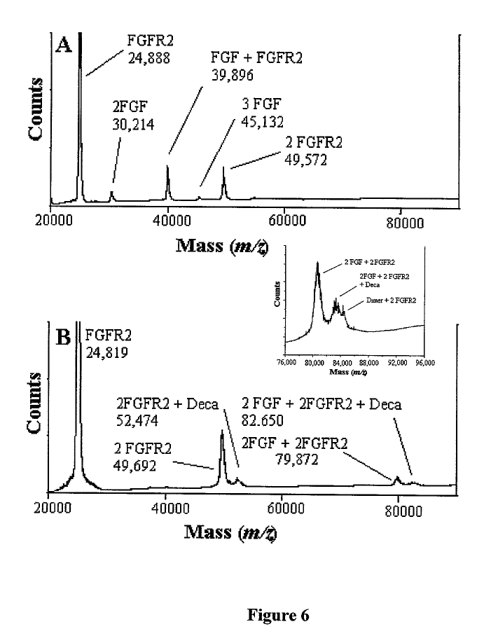

Stoichiometry of FGF2-FGFR-HLGAG interactions—Mass spectrometry was used to determine whether dFGF2 could compete with wild-type FGF2 for binding to FGFR2. Preliminary MALDI analysis of dFGF2 yielded a species consistent with the expected mass for dFGF2 of 37,066 Da. As a next step, we investigated the nature of wild-type FGF2-FGFR interactions both in the presence as well as in the absence of HLGAGs. These studies indicated that, in the absence of an HLGAG, wild type FGF2 bound FGFR with a stoichiometry of 1:1 (FIG. 6(A)), consistent with FGF-FGFR crystal structures (Plotnikov, A. N. et al. (1999) Cell 98(5), 641-50; Plotnikov, A. N. et al. (2000) Cell 101(4), 413-24). However, addition of an HLGAG decasaccharide (consisting of a trisulfated disaccharide repeat unit which is known to bind with high affinity to FGF2 and support FGF2-mediated signaling), resulted in the formation of a detectable 2:2:1 FGF:FGFR:HLGAG complex (FIG. 6(B)), again consistent with the ternary complex observed for FGF1 (Pellegrini, L. et al. (2000) Nature407(6807), 1029-34). Addition of dFGF2 to this complex resulted in the formation of a new 1:2 complex of dFGF2:FGFR (inset in FIG. 6(B)). Notably, we could detect no dFGF:FGFR species with decasaccharide bound. The absence of this species could be either because the complex does not form in solution or that it is not ionized and detected under the conditions of this experiment. In addition, since the ionization efficacies of the various species undoubtedly differ from one another, with the larger species (especially those containing the decasaccharide) being less amenable to ionization than the smaller species, quantitative estimates of the amount of complex formed in this case is not warranted. However, detection of a 1:2 dFGF:FGFR complex indicates that this species does form at protein levels that approximate those present at the cell surface.

Together, these results indicate that (1) one molecule of dFGF2 having protein contact is able to support receptor dimerization, (2) one of the roles for HLGAGs in FGF binding to FGFR is to support FGF and/or FGFR oligomerization and (3) biochemically one mode of FGF oligomerization, and receptor binding involves a dimer with substantial protein-protein contact. To determine whether the complexes observed via mass spectrometry have a biological role, we tested the ability of dFGF2 to signal in several cell-based systems.

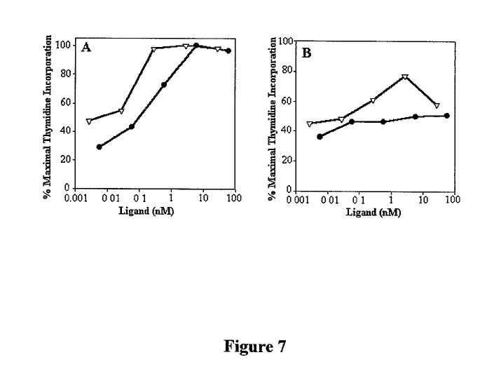

Biological activity of dFGF2—To test if FGF-FGF contacts are involved in signaling, dFGF2 was assayed for its biological activity in the following cell culture assays. Mitogenicity of dFGF2 was tested on SMC treated with or without chlorate. Because chlorate treatment inhibits the biosynthesis of HLGAGs and thereby depletes cell surface HLGAGs, the dependency of HLGAG-binding on the activity of dFGF2 for signaling can be evaluated. With intact cell surface HLGAGs (no chlorate treatment), both wild-type and dFGF2 were active in mediating a proliferative response on SMC (FIG. 7(A)). Importantly, the molar concentrations required to achieve half maximal proliferation by wild-type and dFGF2 were 270 pM and 60 pM, respectively. Hence, dFGF2 exhibited 4.5 folds more activity as compared to wild-type in promoting cell proliferation under these culture conditions. In chlorate-treated SMC, while wild-type only produced a moderate response in proliferation, a marked increase in proliferative response was exhibited by dFGF2, achieving about 80% of full proliferation in HLGAG-depleted cells (FIG. 7(B)). The results from the SMC proliferation assay suggest a higher potency in stimulating proliferation and a lower dependence on HLGAG for signaling by dFGF2.

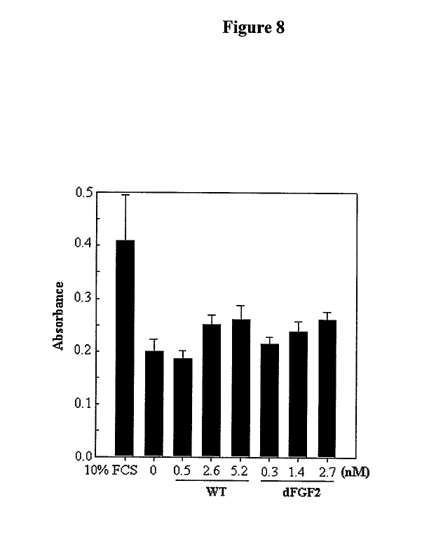

In addition to SMC cells, FGF2 is a potent angiogenic factor well known for its ability to induce cell survival in endothelial cells. Therefore, we determined the ability of dFGF2 to promote cell viability in HUVEC. Using the calorimetric MTS dye that reflects the mitochondrial integrity of viable cells, the HUVEC survival assay provides a sensitive way to measure endothelial cell viability mediated by the growth factors added. In serum deprived HUVEC, cell viability was about 50% of that grown in 10% serum (FIG. 8). Addition of various concentrations of wild-type and dFGF2 can partially recover cell viability in a dose-dependent manner. Again, dFGF2 was more active than wild-type in stimulating survival in HUVEC on a molar basis, consistent with its elevated potency observed in SMC. Together, the biological activity of dFGF2 from two independent cell types demonstrates that the dimeric construct binds to and activates FGFR to elicit various downstream signals as measured by the biological assays.

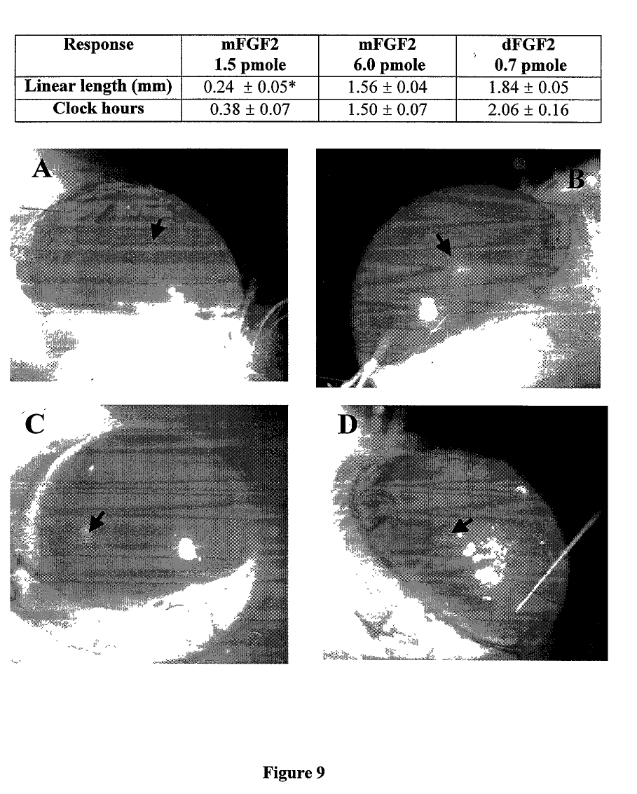

In vivo potency of dFGF2. To extend the above in vitro findings, the ability of dFGF2 to induce angiogenesis in an experimental in vivo model was investigated. The activity of mFGF2 and dFGF2 were compared, side by side, using the rat corneal pocket assay, the results of which are shown in FIG. 8. As anticipated, control pellets containing no FGF2 (i. e. no angiogenic stimuli) failed to induce appreciable angiogenesis (FIG. 9(A)). mFGF2 induced angiogenic response in a dose-dependent manner with little angiogensis induced at a protein level of 1.5 pmole (FIG. 9(B)) and more extensive angiogenesis induced at 6 pmole (FIG. 9(C)). Thus, the extent of angiogenesis induced by mFGF2 is accurately reflected both by the length of induced vessels as well as the circumference of those vessels. Compared to mFGF2, dFGF2 induced more extensive angiogenesis in the corneas of rats at a lower concentration of 0.7 pmole (FIG. 9(D)). With dFGF2, induced blood vessels were longer, of larger circumference, and more plentiful, as measured by “clock hours” or the extent of angiogenesis around the limbus. In fact 0.7 pmole of dFGF2 was a better angiogenic stimulus than was mFGF2 at an 8-fold higher level, viz., 6 pmole. Thus, the biological potency of dFGF2, as measured in in vitro cell culture experiments, was retained in an in vivo animal model, suggesting that the dFGF2 construct is a potent biological mediator.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A composition, comprising a stabilized modified FGF dimer comprising two FGF monomers linked to one another, wherein the dimer includes at least one modification from a native FGF dimer.

2. A pharmaceutical composition, comprising a stabilized modified FGF dimer comprising two FGF monomers linked to one another, wherein the dimer includes at least one modification from a native FGF dimer, and a pharmaceutically acceptable carrier.

3. The pharmaceutical composition of claim 2, further comprising another therapeutic agent.

4. The pharmaceutical composition of claim 2, wherein the composition is sterile.

5. The pharmaceutical composition of claim 2, wherein the dimer includes at least two modifications from a native FGF dimer.

6. The pharmaceutical composition of claim 2, wherein the dimer includes at least five modifications from a native FGF dimer.

7. The pharmaceutical composition of claim 2, wherein the two FGF monomers are FGF2.

8. The pharmaceutical composition of claim 2, wherein the modification is a linker molecule connecting the two monomers.

9. The pharmaceutical composition of claim 8, wherein the linker molecule is a peptide.

10. The pharmaceutical composition of claim 2, wherein the FGF dimer is a protein produced by recombinant DNA technology.

11. The pharmaceutical composition of claim 9, wherein the FGF dimer is a protein produced by expression of a nucleic acid having the sequence of SEQ ID NO.: 5.

12. The pharmaceutical composition of claim 2, wherein at least one FGF monomer has an amino acid sequence corresponding to SEQ ID NO.: 1 or a functionally equivalent variant thereof.

13. The pharmaceutical composition of claim 2, wherein the modification is in at least one of the FGF monomers and is a cysteine residue that does not occur in the native FGF monomer.

14. The pharmaceutical composition of claim 13, wherein at least one FGF monomer has an amino acid sequence corresponding to SEQ ID NO.: 7 or a functionally equivalent variant thereof, but wherein the FGF monomer includes at least one cysteine residue at amino acid number 81 (SEQ ID NO.: 2).

15. The pharmaceutical composition of claim 13, wherein at least one FGF monomer has an amino acid sequence corresponding to SEQ ID NO.: 7 or a functionally equivalent variant thereof, but wherein the FGF monomer includes at least one cysteine residue at amino acid number 100 (SEQ ID NO.: 3).

16. The pharmaceutical composition of claim 13, wherein both FGF monomers have an amino acid sequence corresponding to SEQ ID NO.: 7 or a functionally equivalent variant thereof, but wherein the FGF monomers include at least one cysteine residue at each of amino acid numbers 81 and 100 (SEQ ID NO.: 4).

17. The pharmaceutical composition of claim 16, wherein at least one of the naturally occurring cysteines includes a conservative or non-conservative substitution.

18. The pharmaceutical composition of claim 13, wherein both of the FGF monomers include a cysteine residue that does not occur in the native FGF monomer.

19. The pharmaceutical composition of claim 13, wherein at least one FGF monomer has an amino acid sequence corresponding to SEQ ID NO.: 2.

20. The pharmaceutical composition of claim 13, wherein at least one FGF monomer has an amino acid sequence corresponding to SEQ ID NO.: 3.

21. The pharmaceutical composition of claim 13, wherein at least one FGF monomer has an amino acid sequence corresponding to SEQ ID NO.: 4.

22. The pharmaceutical composition of claim 2, wherein the two FGF monomers are linked to one another by a chemical linkage.

23. The pharmaceutical composition of claim 2, wherein the two FGF monomers are linked to one another by a disulfide bond.

24. The pharmaceutical composition of claim 2, wherein the modification is in at least one of the FGF monomers and is a deletion of at least one of the 9 N-terminal amino acid residues of the monomer.

25. The pharmaceutical composition of claim 24, wherein all 9 of the N-terminal amino acid residues of the monomer are deleted.

26. The pharmaceutical composition of claim 24, wherein both of the FGF monomers include a deletion of at least one of the 9 N-terminal amino acid residues.

27. The pharmaceutical composition of claim 2, wherein the dimer is complexed with an HLGAG.

28. The pharmaceutical composition of claim 9, wherein the peptide linker is selected from the group consisting of GAL, GAR, and GARG.

29. The pharmaceutical composition of claim 9, wherein the peptide linker includes a protease site or an integrin binding sequence, such as RGD.

30. The pharmaceutical composition of claim 24, further comprising a sequence selected form the group consisting of a protease site or an integrin binding sequence at the N-terminal end of the monomer.

31. The pharmaceutical composition of claim 2, wherein the FGF dimer is formulated in a microparticle.

32. An FGF dimer, comprising an FGF dimer composed of two FGF monomers linked to one another via a peptide linker.

33. The FGF dimer of claim 32, further comprising a pharmaceutically acceptable carrier.

34. The FGF dimer of claim 32, wherein the FGF dimer is formulated for delivery to a subject.

35. The FGF dimer of claim 34, wherein the dimer is complexed with an HLGAG.

36. The FGF dimer of claim 32, wherein at least one FGF monomer has an amino acid sequence corresponding to SEQ ID NO.: 1 or a functionally equivalent variant thereof.

37. The FGF dimer of claim 32, wherein the peptide linker is selected from the group consisting of GAL, GAR, and GARG.

38. The FGF dimer of claim 32, wherein the peptide linker includes a protease site or an integrin binding sequence, such as RGD.

39. A method for promoting signal transduction, comprising:
contacting a cell with the FGF dimer of any one of claims 1-30 or 32-36 in an effective amount for promoting signal transduction.

40. A method for treating stroke, comprising:
administering to a subject in need thereof a stabilized FGF dimer composed of two FGF monomers linked to one another and a pharmaceutically acceptable carrier in an effective amount for treating stroke.

41. The method of claim 40, wherein the stabilized FGF dimer the composition of claim 1.

42. A method for treating stroke, comprising:
administering to a subject in need thereof the compositions of an FGF dimer of any one of claims 2-30 or 32-38 in an effective amount for treating stroke.

43. The method of claim 40, wherein the subject is a human.

44. The method of claim 40, further comprising pre-incubating the FGF dimer with an HLGAG prior to administering it to the subject.

45. A method for promoting angiogenesis, comprising:
administering to a subject in need thereof a stabilized FGF dimer composed of two FGF monomers linked to one another and a pharmaceutically acceptable carrier in an effective amount for promoting angiogenesis.

46. The method of claim 45, wherein the stabilized FGF dimer the composition of claim 1.

47. A method for promoting angiogenesis, comprising:
administering to a subject in need thereof the compositions of an FGF dimer of any one of claims 2-30 or 32-38 in an effective amount for promoting angiogenesis.

48. The method of claim 45, wherein the method is a method for promoting wound healing.

49. The method of claim 45, wherein the method is a method for promoting collateral blood vessel formation.

50. The method of claim 45, further comprising pre-incubating the FGF dimer with an HLGAG prior to administering it to the subject.

51. A method for promoting nerve regeneration, comprising:
administering to a subject in need thereof a stabilized FGF dimer composed of two FGF monomers linked to one another and a pharmaceutically acceptable carrier in an effective amount for promoting nerve regeneration.

52. The method of claim 51, wherein the stabilized FGF dimer the composition of claim 1.

53. A method for promoting nerve regeneration, comprising:
administering to a subject in need thereof the compositions of an FGF dimer of any one of claims 2-30 or 32-38 in an effective amount for promoting nerve regeneration.

54. The method of claim 51, further comprising pre-incubating the FGF dimer with an HLGAG prior to administering it to the subject.

55. A method for preventing myocardial damage in heart disease and surgery, comprising:
administering to a subject in need thereof, an effective amount for preventing myocardial damage of a stabilized FGF dimer composed of two FGF monomers linked to one another and a pharmaceutically acceptable carrier.

56. The method of claim 55, wherein the stabilized FGF dimer the composition of claim 1.

57. A method for preventing myocardial damage in heart disease and surgery, comprising:
administering to a subject in need thereof, an effective amount for preventing myocardial damage of the compositions of an FGF dimer of any one of claims 2-30 or 32-38.

58. The method of claim 55, further comprising pre-incubating the FGF dimer with an HLGAG prior to administering it to the subject.

59. A method for treating or preventing nervous system disease, comprising:
administering to a subject in need thereof, an effective amount for treating or preventing nervous system disease a stabilized FGF dimer composed of two FGF monomers linked to one another and a pharmaceutically acceptable carrier.

60. The method of claim 59, wherein the stabilized FGF dimer the composition of claim 1.

61. A method for treating or preventing nervous system disease, comprising:
administering to a subject in need thereof an effective amount for treating or preventing nervous system disease the composition of an FGF dimer of any one of claims 2-30 or 32-38.

62. The method of claim 59, wherein the nervous system disease is a disease of the central nervous system.

63. The method of claim 59, wherein the nervous system disease is a disease of the peripheral nervous system.

64. A screening assay for identifying an FGF dimer binding compound, comprising:
contacting a library of compounds with the FGF dimer of any one of claims 1-25 or 28-34, and identifying a compound that binds the FGF dimer to identify the FGF dimer binding compound.

65. An FGF dimer binding compound identified according to the assay of claim 64.

66. The assay of claim 64, further comprising determining whether the FGF binding compound is an FGF inhibitor by determining whether the FGF binding compound can block FGF dimer interaction with an FGF receptor.

67. An FGF inhibitor identified according to the assay of claim 66.

68. A method for inhibiting FGF activity in a subject by administering to the subject an FGF inhibitor of claim 67.

69. A method for treating cancer, comprising:
administering to a subject in need thereof, an effective amount for treating cancer of the FGF inhibitor of claim 67 and a pharmaceutically acceptable carrier.

70. A method for inhibiting angiogenesis, comprising:
administering to a subject in need thereof, an effective amount for inhibiting angiogenesis of the FGF inhibitor of claim 67 and a pharmaceutically acceptable carrier.

71. A method for treating chronic inflammation, comprising:
administering to a subject in need thereof, an effective amount for treating chronic inflammation of the FGF inhibitor of claim 67 and a pharmaceutically acceptable carrier.

72. A method for treating or preventing an FGF sensistive disorder, comprising:
administering to a subject in need thereof, an effective amount for activating an FGFR the composition of an FGF dimer of any one of claims 2-30 or 32-38.

73. A method for treating or preventing an FGF sensistive disorder, comprising:
administering to a subject in need thereof, an effective amount for activating an FGFR a stabilized FGF dimer composed of two FGF monomers linked to one another and a pharmaceutically acceptable carrier.

74. The method of claim 73, wherein the stabilized FGF dimer the composition of claim 1.

75. A pharmaceutical composition, comprising a modified FGF dimer comprising two FGF monomers linked to one another, wherein the dimer includes at least one modification from a native FGF dimer, and a pharmaceutically acceptable carrier.
